(19) United States
(12) Patent Application Publication
(10) Pub. No.: US 2020/0384118 A1
(43) Pub. Date: Dec. 10, 2020

(54) IMMUNOTOXIN CONJUGATES FOR USE IN THERAPY

(71) Applicant: FRIEDRICH-ALEXANDER-UNIVERSITAT ERLANGEN-NURNBERG, Erlangen (DE)

(72) Inventor: Fabian MÜLLER, Erlangen (DE)

(73) Assignee: FRIEDRICH-ALEXANDER-UNIVERSITAT ERLANGEN-NURNBERG, Erlangen (DE)

(21) Appl. No.: 15/733,285

(22) PCT Filed: Dec. 18, 2018
(86) PCT No.: PCT/EP2018/085503

§ 371 Date: Jun. 19, 2020

(30) Foreign Application Priority Data
Dec. 21, 2017 (EP) 17209379.1

(51) Int. Cl. A61K 47/64; C07K 16/28; C07K 16/32; A61K 47/68

(52) U.S. Cl. CPC A61K 47/6415; C07K 16/2803; C07K 16/2896; C07K 16/2863; C07K 16/32; C07K 2317/66; A61K 47/6811; C07K 2317/55; C07K 2317/622; C07K 2317/624; C07K 2317/565; A61K 47/6849

(57) ABSTRACT

The present invention relates to conjugates comprising an immunotoxin linked to a therapeutic agent, wherein said immunotoxin comprises a binding domain fused to an adenosine diphosphate (ADP) ribosylating toxin, pharmaceutical compositions comprising said conjugates, and methods for the preparation of said immunotoxins.

### FIELD OF THE INVENTION

The present invention relates to a conjugate comprising an immunotoxin linked to a therapeutic agent, wherein said immunotoxin comprises a binding domain fused to an adenosine diphosphate (ADP) ribosylating toxin. The present invention further relates to the conjugate of the present invention for use in a method of treating cancer and autoimmune diseases. Moreover, the present invention also provides for a pharmaceutical composition comprising the conjugate of the present invention. The present invention also refers to a nucleic acid encoding the immunotoxin according to the present invention, a vector comprising said nucleic acid and a host cell comprising said nucleic acid. The present invention further relates to a method for the preparation of the conjugate of the present invention.

### BACKGROUND OF THE INVENTION

Cancer is one of the leading causes of morbidity and mortality worldwide, with approximately 14 million new cases in 2012. The number of new cases is expected to rise by about 70% over the next 2 decades. Cancer is the second leading cause of death globally, and was responsible for 8.8 million deaths in 2015. Globally, nearly 1 in 6 deaths is due to cancer. Among chemotherapy and radiotherapy, immunotherapy has become a standard in cancer treatment. Antibodies are used in this respect to deliver therapeutic payloads to target cancer cells, e.g. in form of antibody-drug conjugates (ADCs) or recombinant immunotoxins (RITs).

ADCs consist of an antibody structure chemically attached to highly toxic chemotherapeutic agents, thereby specifically targeting a highly cytotoxic drug to the tumor (but limiting its deposition elsewhere), which increases antitumor activity but decreases systemic toxicity. All ADCs currently approved by the U.S. Food and Drug Administration (FDA) and most of these in development use anti-microtubule chemotherapeutics. The CD22-targeting ADC Pinatuzumab vedotin (PV) consists of an antibody conjugates with monomethyl autistin E (MMAE) which is a small molecule that targets microtubules. Microtubules (MTs), made up of polymerized α- and β-tubulin dimers, form the mitotic spindle crucial for the separation of chromosomes during cell division. Equally, MTs are very important for directional intracellular transport of vesicles, proteins, and mRNA, serve important roles in cytoskeleton formation, endothelial cell adhesion, migration, and cell-to-cell interaction. Once inside the cell, antitubulin drugs disrupt microtubule organization or alter microtubule dynamics, leading to mitotic arrest and cell death (Jordan et al. 2004, Nat. Rev. Cancer; 4:253-265). Therefore, MTs represent a validated primary oncologic target of MT-binding agents in humans and a number of antitubulin agents are attractive options for antibody drug conjugates. Because of their greatly improved therapeutic window, ADCs have considerably developed and have proven effective against some refractory tumors.

Recombinant immunotoxins (RIT) combine the specificity of an antibody with some of nature's most toxic proteins. RITs are fusion proteins that consist of bacterial or plant toxins fused to a targeting moiety. The targeting domain is most commonly the antibody fragment of a monoclonal antibody. The RIT binds to the specific antigen on the target cell via the antibody, it is internalized and kills the cell by arresting protein synthesis (Pastan et al. 2006, The Oncologist; 20:176-185). The initial immunotoxins were made of full-length antibodies coupled with plant toxins like ricin or gelonin. Subsequently, bacteria protein toxins like Pseudomonas exotoxin (PE) and diphtheria toxin were used. Said toxins comprise an enzyme that ADP-ribosylates the eukaryotic elongation factor-2 (eEF-2), leading to an arrest of protein biosynthesis and apoptosis (Pastan et al. 2006).

The first generation of immunotoxins were made of a full length PE protein attached to whole monoclonal antibodies and showed severe side effects due to binding of the drug to the physiological PE receptor (Pastan et al. 2006, Nat. Rev. Cancer; 6:599-565). Second generation immunotoxins were made by applying chemical conjugation, i.e. regions not essential for cell killing were removed from the toxin to eliminate the physiologic PE tropism, and replaced by a specific antibody to specifically deliver PE to target cells. These immunotoxins could not bind to normal cells, but their entry into tumors was still limited by a large molecular size and caused side effects such as vascular leak syndrome (VLS), hemolytic uremic syndrome, and pleuritis. Third generation immunotoxins have been further improved and were made by recombinant DNA technology. Using molecular cloning techniques, immunotoxins including Fv fragments of antibodies attached to enzymatic active toxin domains could be produced (Allahyari et al., Tumor Biology 2017; 1-11). Immunotoxins kill cancer cells in the low pico-molar range in vitro, but despite this high in vitro activity, efficacy in clinical trials is unexpected lower. Moreover, immunotoxins are highly immunogenic to human and less immunogenic variants had to be prepared using site-directed mutagenesis of toxin genes, resulting in an improved activity and reduced off-target toxicity (Onda et al., J. Immunol 2006; 177(12):8822-8834).

Immunotoxins have several favorable properties not shared by ADCs. The unique mechanism of action of immunotoxins combines little cross-resistance with other active substances and a non-overlapping toxicity profile, allowing for a combination with standard of care agents. Additionally, ADCs may cause off-target toxicity due to inappropriate payload dissociation, while third generation immunotoxins are based on peptide linkers, which require a specific intracellular protease to unlink.

Targeted therapy like the immunotoxins is an emerging treatment modality of cancer therapy which may replace some of the conventional therapies in the future. Following the same principle, specifically targeting surface markers is also a promising strategy in the treatment of autoimmune diseases. The application of immunotoxins for the treatment of rheumatoid arthritis and psoriasis showed promising results in vitro and in first clinical trials (Van Roon et al. 2003, Arthrit Rheum; 48(5): 1229-1238; LeMaistre 2000, Clin Lymphoma; 1:S37-S40).

Several Immunotoxins have been clinically tested and showed promising activity. Because of their short serum half-life of 20 minutes (Wayne et al. Blood 2017), efficacy of immunotoxins was improved substantially by continuous infusion showing that the longer exposure to target cells is necessary for cell killing (Muller et al. 2016, Clin. Cancer Res.; 22:4913-4922; Muller et al. 2017, Oncotarget; 8:30644-30655). Immunotoxins may further be used in cancer therapy as part of a combination therapy comprising other cytostatic agents. However, finding cytostatics suitable in this respect is ongoing and challenging. In sum, there is an unmet need to further improve immunotoxin-based therapies by optimizing activity with the least possible side effects. The technical problem underlying the present application is thus to comply with this need. The technical problem is solved by providing the embodiments reflected in the claims, described in the description and illustrated in the examples and figures that follow.

### SUMMARY OF THE INVENTION

The present invention is, at least partly, based on the surprising finding that conjugates comprising an immunotoxin linked to a therapeutic agent show improved activity and reduced toxicity, i.e. are well suited for therapeutic purposes. In particular, the present invention provides for conjugates where an immunotoxin comprising a binding domain such as an antibody or functional fragment thereof fused to an adenosine diphosphate (ADP) ribosylating toxin has been linked to a small molecule therapeutic agent, preferably having cytostatic and/or cytotoxic activity. More specifically, the inventors of the present invention managed to conjugate a chemotherapeutic agent such as myatansinoid (DM1) and Pseudomonas exotoxin (PE) A to a monovalent antibody fragment, resulting in highly specific and therapeutically active compounds called “Duotoxins”, which can be used to selectively kill target cells, in particular tumor cells. Accordingly, the conjugates of the present invention allow for various applications in the treatment of cell-based diseases like cancer or autoimmune diseases.

When combining ADP-ribosylating toxins and chemotherapeutics on one antibody molecule, the produced constructs allow for a simultaneous delivery of two synergistically acting drugs to a target cell, thereby taking advantage of the combination therapy while reducing side effects. In this respect, the therapeutic efficacy can be optimally exploited and adverse events are significantly reduced.

Previous approaches individually used immunoglobulin-based conjugates with either a protein toxin (immunotoxin) or a chemotherapeutic agent (ADC), and immunotoxins have been found to be synergistically enhanced by systemically administered free microtubule-targeting cytostatic agents such as paclitaxel (Müller et al. 2017, Oncotarget; 8:30644-30655). The combined usage of both properties—toxin and chemotherapeutic agent—coupled to one single molecule has never been described or suggested before. Even though paclitaxel can by coupled to sugar structures (Lee Bioconjug Chem. 2008 June; 19(6):1319-25) or PEG (nab-paclitaxel), a conjugation method that results in serum stable linkage for the use in patients has not yet been developed. Contrary to said prejudice, the inventors of the present invention reached to combine both an ADP-ribosylating toxin and a microtubule-targeting agent on one targeting protein without significant loss of effectivity. This was unforeseeable, as immunotoxins and ADCs have exclusively been described in the art as comprising toxin variants or chemotherapeutic drugs, but no combination thereof at one and the same polypeptide. The solution provided by the present invention is however remarkable, since the synergistic effect of the two drugs is maintained when compared to an individual application, while systemic side effects are likely reduced.

The conjugates of the present invention are constructed to comprise both drugs linked to a targeting protein. Therefore, a suitable immunotoxin for the conjugation with a therapeutic drug had to be generated. Said immunotoxin preferably comprises the ADP-ribosylating toxin PE comprising from the amino terminus a furin cleavage site, a catalytically active domain of the toxin, wherein all lysine residues are substituted by any other amino acid, and a modified C-terminal REDLK motif. Said toxin can be a modified Pseudomonas exotoxin (PE) A as shown in FIG. 2, which allows for an amid-directed coupling to a binding domain without reduction of the PE activity. In addition, the conjugates of the present invention comprise a microtubule-targeting therapeutic active small molecule, preferably mertansine and monomethyl auristatin (MMA), which target the tip of microtubules which suppress microtubule dynamics, resulting in mitotic arrest and cell death.

The Duotoxins described herein are active in vitro and have similar or better activity against cancer cell lines when compared to immunotoxins not comprising the therapeutic drug and kill the target cells in a dose-dependent manner (FIG. 3). In addition, Duotoxins according to the present invention show time-dependent effects in vitro, i.e. activity of the conjugates depends on the time cells are exposed (FIG. 4). In fact, Duotoxins are more active the longer a target cell is exposed, suggesting that Duotoxins will be even more active when continuously administered instead of an administration by bolus doses (FIG. 4). Further, the Duotoxins described herein show the intended synergy and have improved activity against cancer cell lines in vitro and in xenograft animal models when compared to ADCs or normal immunotoxins (FIG. 5). In comparison with a combination of an immunotoxin and a chemotherapeutic agent as described in the art, the Duotoxins of the present invention show no significant loss of impact, but allow for an “all-in-one” application. The chemotherapeutic agent however, is not given systemically likely reducing unspecific side effects. An alternative administration of conventional ADCs in combination with conventional immunotoxins would compete for the binding to the target antigen and therefore reduce each other's activity competitively. In sum, the present invention impressively demonstrates that the novel Duotoxin conjugates described herein successfully deliver synergistic drugs simultaneously to a target cell, and thus, provide a new tool for targeted cell therapy.

Although enhancement of immunotoxin effects by microtubule-targeting compounds like paclitaxel has been described in vivo in xenograft models, the synergy of immunotoxins and paclitaxel in vivo is contrasted by a small or absent synergy in vitro, supporting an in vivo specific mechanism. In addition, linkage of paclitaxel to immunoglobulins is associated with a partial loss of activity. However, using the Duotoxins of the present invention comprising both, an ADP-ribosylating toxin and a microtubule-targeting paclitaxel-like small molecule, highly synergistic effects could be demonstrated in vitro and in vivo when compared with state of the art immunotoxins or antibody drug conjugates (FIG. 5). In this respect, bolus doses of Duotoxins were significantly better in vivo than either immunotoxin or ADC alone. In sum, the new immunotoxin conjugates of the present invention maintain or even enhance the strong synergistic effects of microtubule-targeting agents when combined with immunotoxin, while healthy cells are not affected and side effects are likely reduced.

Accordingly, the present invention demonstrates that the immunotoxin conjugates disclosed herein have a greatly improved therapeutic effect due to a strong synergy of the two drugs comprised and are superior to combination therapies comprising immunotoxins and unconjugated chemotherapeutic agents which are not specifically delivered and therefore cause unspecific side effects. The immunotoxins used in this regard are defined as recombinant fusion proteins of a structure that conveys binding to a specific target structure and the catalytic active parts of an ADP-ribosylating toxin. The present invention is further based on the discovery that particularly immunotoxins comprising the ADP-ribosylating toxin PE that comprises a furin cleavage site, a catalytically active domain of the toxin that lacks all lysine residues and has a modified C-terminal REDLK-motive are particularly useful in this respect and constitute suitable starting compounds for the chemical conjugation of microtube-targeting small molecules to said immunotoxins, without resulting in a reduced PE activity. However, also other catalytic domains of ADP-ribosylating bacterial toxins such as diphtheria toxin comprising a catalytically active domain that ADP-ribosylates EF2 and is engineered without lysine residues can be used according to the present invention. The Duotoxins of the present invention are significantly more active than either the drug or the immunotoxin alone, underlying that Duotoxins successfully deliver synergistic drugs simultaneously to a target cell.

Accordingly, on a general basis, the present invention relates to means and methods for targeted cell therapy, thereby reducing side effects of combined therapeutic approaches presently known in the art. In particular, the present invention aims at the treatment of cancer and autoimmune disease in a specific, more efficient manner, thereby using new targeting conjugates. In sum, the present invention provides for new immunotoxin conjugates called “Duotoxins” comprising an immunotoxin and a therapeutic small molecule combined on one antibody molecule, thereby allowing for a simultaneous delivery of the two synergistically acting drugs to a target cell without loss of activity of either of the drug compounds.

Thus, in a first aspect, the present invention relates to a conjugate comprising an immunotoxin linked to a therapeutic agent, wherein said immunotoxin comprises a binding domain fused to an adenosine diphosphate (ADP) ribosylating toxin. In particular, it is envisaged that the ADP-ribosylating toxin is Pseudomonas exotoxin (PE) A or diphtheria toxin.

The binding domain fused to an adenosine diphosphate (ADP) ribosylating toxin is preferably an antibody or a functional fragment thereof. Said functional fragment is preferably selected from the group consisting of an antibody binding fragment Fab, a disulfide-stabilized Fv (dsFv), or a single-chain Fv (scFv).

It is envisaged that the binding domain fused to an adenosine diphosphate (ADP) ribosylating toxin is capable of binding to a cell surface protein, preferably CD22, mesotheline, fms like tyrosine kinase 3 (FLT-3), HER2/neu or CD138.

Said binding domain preferably comprises a VL region comprising CDR-L1, CDR-L2 and CDR-L3 and/or a VH region comprising CDR-H1, CDR-H2 and CDR-H3 selected from:
(a) CDR-L1 as shown in SEQ ID NO: 1, CDR-L2 as shown in SEQ ID NO: 2 and CDR-L3 as shown in SEQ ID NO: 3 and/or CDR-H1 as shown in SEQ ID NO: 4, CDR-H2 as shown in SEQ ID NO: 5 and CDR-H3 as shown in SEQ ID NO: 6;
(b) CDR-L1 as shown in SEQ ID NO: 7, CDR-L2 as shown in SEQ ID NO: 8 and CDR-L3 as shown in SEQ ID NO: 9 and/or CDR-H1 as shown in SEQ ID NO: 10, CDR-H2 as shown in SEQ ID NO: 11 and CDR-H3 as shown in SEQ ID NO: 12;
(c) CDR-L1 as shown in SEQ ID NO: 13, CDR-L2 as shown in SEQ ID NO: 14 and CDR-L3 as shown in SEQ ID NO: 15 and/or CDR-H1 as shown in SEQ ID NO: 16, CDR-H2 as shown in SEQ ID NO: 17 and CDR-H3 as shown in SEQ ID NO: 18; and
(d) CDR-L1 as shown in SEQ ID NO: 19, CDR-L2 as shown in SEQ ID NO: 20 and CDR-L3 as shown in SEQ ID NO: 21 and/or CDR-H1 as shown in SEQ ID NO: 22, CDR-H2 as shown in SEQ ID NO: 23 and CDR-H3 as shown in SEQ ID NO: 24.

It is also envisaged that the said binding domain comprises a VL region and/or a VH region selected from:
(a) a VL region as shown in SEQ ID NO: 25 and/or a VH region as shown in SEQ ID NO: 26;
(b) a VL region as shown in SEQ ID NO: 27 and/or a VH region as shown in SEQ ID NO: 28;
(c) a VL region as shown in SEQ ID NO: 29 and/or a VH region as shown in SEQ ID NO: 30; and
(d) a VL region as shown in SEQ ID NO: 31 and/or a VH region as shown in SEQ ID NO: 32.

The therapeutic agent comprised by the conjugate of the present invention has preferably cytostatic and/or cytotoxic activity. Said therapeutic agent may be an anti-cancer drug, preferably a microtubule-targeting drug. Said drug may be selected from mertansine and monomethyl auristatin (MMA). Said mertansine is preferably myatansinoid (DM1). Said monomethyl auristatin is preferably monomethyl auristatin E (MMAE) or monomethyl auristatin D (MMAD).

It is envisaged that at least one molecule of the therapeutic agent, preferably 2, 3, 4, 5, 6, 7, or 8 molecules of the therapeutic agent, are linked to one immunotoxin molecule.

In particular, it is envisaged that the conjugate comprising an adenosine diphosphate (ADP) ribosylating toxin comprises from the amino terminus:
(i) a furin cleavage site, preferably comprising an amino acid sequence as shown in SEQ ID NO: 33 or an amino acid sequence having at least 85% identity to the amino acid sequence as shown in SEQ ID NO: 33 and the amino acid in position 6 is an arginine,
(ii) a catalytic domain, preferably comprising an amino acid sequence as shown in SEQ ID NO: 34 or an amino acid sequence having at least 85% identity to the amino acid sequence as shown in SEQ ID NO: 34, wherein lysine residues in the catalytic domain have been substituted by any other amino acid, and
(iii) the amino acid sequence as shown in SEQ ID NO: 36, preferably as shown in SEQ ID NO: 37, or
(i) a catalytic domain, preferably comprising an amino acid sequence as shown in SEQ ID NO: 35 or an amino acid sequence having at least 85% identity to the amino acid sequence as shown in SEQ ID NO: 35, wherein lysine residues in the catalytic domain have been substituted by any other amino acid.

Preferably, the conjugate of the present invention comprises an adenosine diphosphate (ADP) ribosylating toxin comprising the amino acid sequence as shown in SEQ ID NO: 38.

In view of the present invention, the therapeutic agent may be linked to the immunotoxin by a linker. Said linker is preferably a serum-stable linker. In this regard it is envisages that myatansinoid (DM1) is linked to the immunotoxin by succinimidyl 4-(N-maleimidomethyl)cyclohexane-1-carboxylate (SMCC). It is further envisaged that monomethyl auristatin E (MMAE) and monomethyl auristatin D (MMAD) are linked to immunotoxin via the serum stable linker OSu-Glu-vc-PAB and the myatansinoid (DM1) via SMCC-linker.

According to another aspect, it is intended to use the conjugates of the present invention in a method of treating a subject suffering from a cell-based disease, preferably cancer or an autoimmune disease. Preferably, said cancer is selected from the group consisting of lymphoma, preferably B-cell malignancies, leukemia, ovarian cancer, breast cancer, lung cancer, prostate cancer, colon cancer, kidney cancer, pancreatic cancer, mesothelioma, lymphoma, liver cancer, urothelial cancer, stomach cancer, and cervical cancer. The autoimmune disease is preferably a B-cell mediated disease, preferably any one selected from the group consisting of psoriasis, rheumatoid arthritis, multiple sclerosis, Sjögren's syndrome, and Guillain-Barré syndrome. The subject is preferably a mammal. Said mammal can be a mouse, a rat, a guinea a pig, a rabbit, a cat, a dog, a monkey, a horse or a human, preferably a human.

It is envisaged that the use of the conjugates of the present invention is for treating a subject suffering from cancer or an autoimmune disease comprises administering a therapeutically efficacious amount of said conjugate to the subject. Preferably, said conjugate is continuously administered. The conjugate may be administered intratumorally, parenterally, subcutaneously, intravenously, intramuscularly, intraperitoneally, by intracavitary, intraarterially, transdermally, by application to mucous membranes, intrathecally, or intraarticulary. It is envisaged that the conjugate is administered as an adjunct therapy to surgery and/or radiotherapy. It is further envisaged that the conjugate is administered in combination with a compound used to treat cancer or autoimmune diseases.

In another aspect, the present invention provides for a nucleic acid encoding an immunotoxin comprising a binding domain fused to an adenosine diphosphate (ADP) ribosylating toxin, wherein said ADP ribosylating toxin comprises from the amino terminus:
(i) a furin cleavage site, preferably comprising an amino acid sequence as shown in SEQ ID NO: 33 or an amino acid sequence having at least 85% identity to the amino acid sequence as shown in SEQ ID NO: 33 and the amino acid in position 6 is an arginine,
(ii) a catalytic domain, preferably comprising an amino acid sequence as shown in SEQ ID NO: 34 or an amino acid sequence having at least 85% identity to the amino acid sequence as shown in SEQ ID NO: 34, wherein lysine residues in the catalytic domain have been substituted by any other amino acid, and
(iii) the amino acid sequence as shown in SEQ ID NO: 36, preferably as shown in SEQ ID NO: 37, or
(i) a catalytic domain, preferably comprising an amino acid sequence as shown in SEQ ID NO: 35 or an amino acid sequence having at least 85% identity to the amino acid sequence as shown in SEQ ID NO: 35, wherein lysine residues in the catalytic domain have been substituted by any other amino acid.

Preferably, the ADP ribosylating toxin comprises an amino acid sequence as shown in SEQ ID NO: 38. The binding domain encoded by said nucleic acid is preferably an antibody or functional fragment thereof. Preferably, said functional fragment is an antibody binding Fab, a disulfide-stabilized Fv (dsFv), or a single-chain Fv (scFv). Said binding domain is capable of binding to a cell surface protein, preferably CD22, mesotheline (MSLN), fms like tyrosine kinase 3 (FLT-3), HER2/neu or CD138.

The binding domain encoded by said nucleic acid preferably comprises a VL region comprising CDR-L1, CDR-L2 and CDR-L3 and/or a VH region comprising CDR-H1, CDR-H2 and CDR-H3 selected from:
(a) CDR-L1 as shown in SEQ ID NO: 1, CDR-L2 as shown in SEQ ID NO: 2 and CDR-L3 as shown in SEQ ID NO: 3 and/or CDR-H1 as shown in SEQ ID NO: 4, CDR-H2 as shown in SEQ ID NO: 5 and CDR-H3 as shown in SEQ ID NO: 6;
(b) CDR-L1 as shown in SEQ ID NO: 7, CDR-L2 as shown in SEQ ID NO: 8 and CDR-L3 as shown in SEQ ID NO: 9 and/or CDR-H1 as shown in SEQ ID NO: 10, CDR-H2 as shown in SEQ ID NO: 11 and CDR-H3 as shown in SEQ ID NO: 12;
(c) CDR-L1 as shown in SEQ ID NO: 13, CDR-L2 as shown in SEQ ID NO: 14 and CDR-L3 as shown in SEQ ID NO: 15 and/or CDR-H1 as shown in SEQ ID NO: 16, CDR-H2 as shown in SEQ ID NO: 17 and CDR-H3 as shown in SEQ ID NO: 18; and
(d) CDR-L1 as shown in SEQ ID NO: 19, CDR-L2 as shown in SEQ ID NO: 20 and CDR-L3 as shown in SEQ ID NO: 21 and/or CDR-H1 as shown in SEQ ID NO: 22, CDR-H2 as shown in SEQ ID NO: 23 and CDR-H3 as shown in SEQ ID NO: 24.

It is also envisaged that the said binding domain comprises a VL region and/or a VH region selected from:
(a) a VL region as shown in SEQ ID NO: 25 and/or a VH region as shown in SEQ ID NO: 26;
(b) a VL region as shown in SEQ ID NO: 27 and/or a VH region as shown in SEQ ID NO: 28;
(c) a VL region as shown in SEQ ID NO: 29 and/or a VH region as shown in SEQ ID NO: 30; and
(d) a VL region as shown in SEQ ID NO: 31 and/or a VH region as shown in SEQ ID NO: 32.

It is envisaged that the nucleic acid encoding an immunotoxin further comprises a restriction sites flanking the nucleic acid sequences encoding for Fv and Fc. Preferably said restriction site between the nucleic acid sequences encoding for Fv and Fc is a NheI, a NdeI, or a HindIII restriction site.

Also provided herein is a vector comprising the nucleic acid encoding an immunotoxin comprising a binding domain fused to an adenosine diphosphate (ADP) ribosylating toxin. Said vector may further comprise a regulatory sequence, which is operably linked to said nucleic acid sequence. Preferably, said vector is an expression vector. More preferably, said vector is a plasmid.

Further provided is a host cell transformed with a vector comprising the nucleic acid encoding an immunotoxin comprising a binding domain fused to an adenosine diphosphate (ADP) ribosylating toxin. Preferably, said host cell is a prokaryotic or eukaryotic cell. More preferably, said prokaryotic host cell is E. coli.

According to another aspect, the present invention provides for a method for the preparation of the conjugate according to the present invention comprising an immunotoxin linked to a therapeutic agent, wherein said immunotoxin comprises a binding domain fused to an adenosine diphosphate (ADP) ribosylating toxin, comprising cloning a nucleic acid encoding for a fusion protein comprising a binding domain and an ADP ribosylating toxin into an expression vector, transforming a host cell with said expression vector, cultivating said transformed host cell in a nutrient medium, expressing the fusion protein, extracting the fusion protein from said host cell or medium, and conjugating the immunotoxin fusion protein by a linker to a therapeutic agent.

Also provided is a pharmaceutical composition comprising the conjugate according to the present invention comprising an immunotoxin linked to a therapeutic agent, wherein said immunotoxin comprises a binding domain fused to an adenosine diphosphate (ADP) ribosylating toxin. It is envisaged that the pharmaceutical composition further comprises a pharmaceutically acceptable excipient or carrier.

Further provided is a method for the treatment of a subject suffering from cancer or an autoimmune disease, the method comprising administering a therapeutically effective amount of the conjugate of the present invention comprising an immunotoxin linked to a therapeutic agent, wherein said immunotoxin comprises a binding domain fused to an adenosine diphosphate (ADP) ribosylating toxin, to a subject in need thereof. The therapeutically effective amount is preferably sufficient to alleviate or heal said cancer or said autoimmune disease.

Also provided is the use of a compound for the preparation of a medicament for treatment of a subject suffering from cancer or an autoimmune disease, wherein said compound is selected from the conjugate according to the present invention comprising an immunotoxin linked to a therapeutic agent, wherein said immunotoxin comprises a binding domain fused to an adenosine diphosphate (ADP) ribosylating toxin.

### BRIEF DESCRIPTION OF THE DRAWINGS

FIG. 1: Buildup of immunotoxins and Duotoxins. (A) The Pseudomonas exotoxin A (left panel, bottom) contains a receptor binding domain, a transport-related domain II, and the catalytically active domain III. In state of the art immunotoxin, the receptor-binding domain is exchanged with the antigen-binding, variable fragment Fv of an antibody resulting in the final immunotoxin. (B) Duotoxin conjugates according to the present invention comprise an ADP-ribosylating toxin, preferably Pseudomonas exotoxin A, which lacks domain II, is free of lysine residues in the catalytic domain III and is fused to a Fab binding domain, as well as one or more therapeutic drugs such as DM1 linked to the binding domain.

FIG. 2: Structure of immunotoxin conjugates. (A) A schematic describing the various immunotoxins, LR=lysozyme resistant, LMIT=short for lab of molecular immune-therapeutics, X* for any number whereas the antigen-defining Fv may be exchanged to any Fv by “cut and paste” cloning using the newly introduced NheI-restriction site. (B) LMIT-3 by itself is not cytotoxic at high concentrations, but it can block various concentrations of LMIT-2 in a dose-dependent manner.

FIG. 3: Activity of Duotoxins in vitro. (A) LMIT-2 and the Duotoxin-variant LMIT-2-DM1 have identical activity against most cell lines like KOPN-8 in vitro. Mino is a representative of cells which cannot be killed to 100% by LMIT-2, whereas LMIT-2-DM1 kills all cells at high concentrations. DOHH-2 is resistant to the PE-only LMIT-2, the Duotoxin can kill DOHH-2 cells in a dose-dependent manner. (B) The graph shows the molar activity against many cell lines of the IgG-based anti-CD22-MMAE Pinatuzumab vedotin, LMIT-2, and LMIT-3-DM1. Similar color/symbol shows the same cell line, not further specified cells are shown as black circle.

FIG. 4: Activity of Duotoxins increases over exposure period. The indicated cell lines were exposed to a lethal dose of LMIT-3-DM1 for various times, washed, and analyzed for cell death three days after the start of the assay. Cell viability was determined by flow cytometry. Viability decreases, the longer cells are exposed to LMIT-3-DM1.

FIG. 5: Duotoxins are more active than immunotoxin or ADC in the systemic JeKo-1 xenograft model. (A) NSG mice were injected with 5 million JeKo-1 cells at day 1, a few mice analyzed for JeKo-1 BM infiltration at treatment start (Day 14), and the remainder treated with 3 doses of the indicated drug and dose. 2 days after the last treatment, mice were sacrificed and analyzed for JeKo-1 bone marrow infiltration. (B) Equimolar immunotoxin was conjugated either with 200 nmol DM1 or with 200 nmol MMAE under identical conditions and 0.8 mg/kg of respective duotoxin or the immunotoxin only control was administered from day 14 QOD. Mice were sacrificed at day 21 and analyzed for JeKo-1 bone marrow infiltration.

### DEFINITIONS

Unless otherwise stated, the following terms used in this document, including the description and claims, have the definitions given below.

Those skilled in the art will recognize, or be able to ascertain, using not more than routine experimentation, many equivalents to the specific embodiments of the invention described herein. Such equivalents are intended to be encompassed by the present invention.

It is to be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the methods and uses described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “consisting”, “consisting of” and “consisting essentially of” may be replaced with either of the other two terms.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or” as used herein.

As described herein, “preferred embodiment” means “preferred embodiment of the present invention”. Likewise, as described herein, “various embodiments” and “another embodiment” means “various embodiments of the present invention” and “another embodiment of the present invention”.

The word “about” as used herein refers to a value being within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. The term “about” is also used to indicate that the amount or value in question may be the value designated or some other value that is approximately the same. The phrase is intended to convey that similar values promote equivalent results or effects according to the invention. In this context “about” may refer to a range above and/or below of up to 10%. The word “about” refers in some embodiments to a range above and below a certain value that is up to 5%, such as up to up to 2%, up to 1%, or up to 0.5% above or below that value. In one embodiment “about” refers to a range up to 0.1% above and below a given value.

Several documents are cited throughout the text of this disclosure. Each of the documents cited herein (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material. Nothing herein is to be construed as an admission that the invention is not entitled to antedate such disclosure by virtue of prior invention.

The following description includes information that may be useful in understanding the present invention. It is not an admission that any of the information provided herein is prior art or relevant to the presently claimed inventions, or that any publication specifically or implicitly referenced is prior art.

### DETAILED DESCRIPTION OF THE INVENTION

The present invention provides for new immunotoxin conjugates called “Duotoxins”, wherein two distinct payloads have been combined to the same antibody molecule. Said Duotoxins comprise an adenosine diphosphate (ADP) ribosylating toxin, preferably Pseudomonas exotoxin (PE) A, fused to an antibody or antibody fragment and a chemically conjugated therapeutic agent, preferably a microtubule-targeting agent. Thus, the present invention combines two highly synergistic drugs on one antibody molecule, whereas the exceptional synergy of the two drugs allows for a greatly improved therapeutic window.

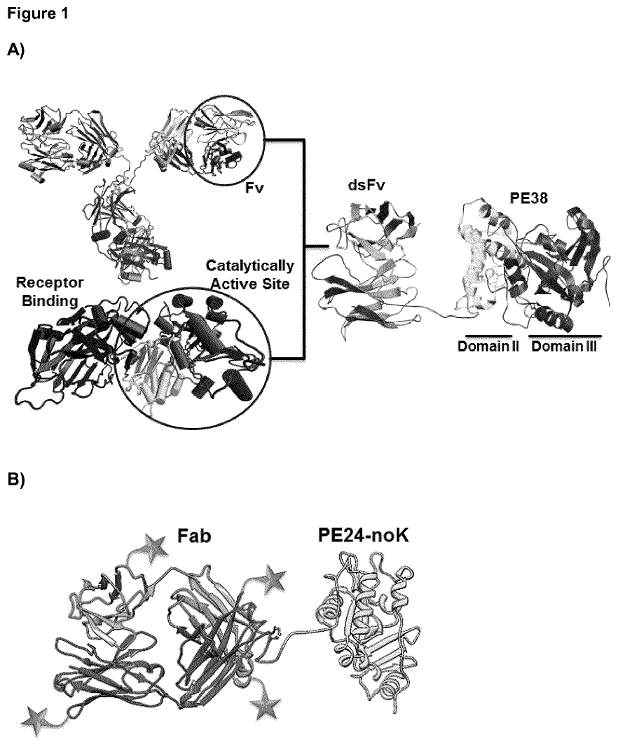

As previously described in the art, immunotoxins are fusion proteins originally comprising an antibody and a bacterial toxin such as Pseudomonas exotoxin A, which comprises a binding domain, a translocation domain, and a catalytically active site (see FIG. 1). The first immunotoxin consisted of the wild-type PE molecule chemically linked to an IgG-antibody targeting the PE to a specific surface receptor. However, it has been known for a long that immunotoxins comprising full length PE lead to severe side effects in treated patients due to binding of the drug to the physiologic PE receptor. In fact, in newer generations of immunotoxins the receptor binding domain has been exchanged with the variable domain of an antibody (Fv) to eliminate the physiologic PE tropism and to specifically deliver PE to the target cell. After binding to the specific surface receptor, immunotoxins are internalized and are transported through various intracellular compartments to the cytosol. PE then ADP-ribosylates the elongation factor 2 (EF2) at the diphthamide residue thereby arresting protein synthesis and inducing apoptosis. The wild type Pseudomonas exotoxin A sequence according to Uniprot accession number P 11439 is shown in SEQ ID NO: 39. The receptor binding domain corresponds to amino acids 1 to 252 of SEQ ID NO: 39, the translocation domain corresponds to amino acids 253 to 364 of SEQ ID NO: 39, and the catalytic domain corresponds to amino acids 365 to 613 of SEQ ID NO: 39. The retrograde trafficking of PE depends on a C-terminal KDEL-motive, and without the KDEL-motive PE is supposed to be inactive. The exact function of domain II is not understood, the average activity of immunotoxins lacking domain II, however, increases by two-fold. Immunotoxins kill cancer cells in the low pico-molar range in vitro. Despite this high in vitro activity, efficacy of immunotoxins in clinical trials was unexpectedly low.

As states above, the present invention combines two highly synergistic drugs on one targeting protein, whereas the synergy of the two drugs leads to improved therapeutic effects and, thus, provides for a new and promising cell-directed therapy. The conjugates of the present invention are based on the known efficacy of bacterial or plant toxin to kill target cell by the enzymatic activity and ADP-ribosylation of the eukaryotic elongation factor-2 (eEF-2). In this respect the conjugates described herein benefit from the known non-overlapping toxicity profile and low cross-resistance of said toxins with other active substances, allowing for a combination with small molecule drugs. On the other side, the efficiency of the immunotoxin-based conjugates of the present invention is attributed to the drug binding site on microtubules. Although effects of chemotherapeutics on microtubules are known, the microtubule-targeting compounds such as mertansine and monomethyl auristatin used according to the present invention seem to be directed to a unique microtubules binding site. Further, while paclitaxel can be directly applied, single application of mertansine and monomethyl auristatin leads to toxic effects, rather excluding said microtube-targeting compounds for combined immunotoxin therapy. However, fusion of mertansine and monomethyl auristatin to immunotoxins comprising ADP-ribosylating toxins resulted in the highly efficient and therapeutic active new immunotoxin conjugates for the treatment of cancer and autoimmune diseases.

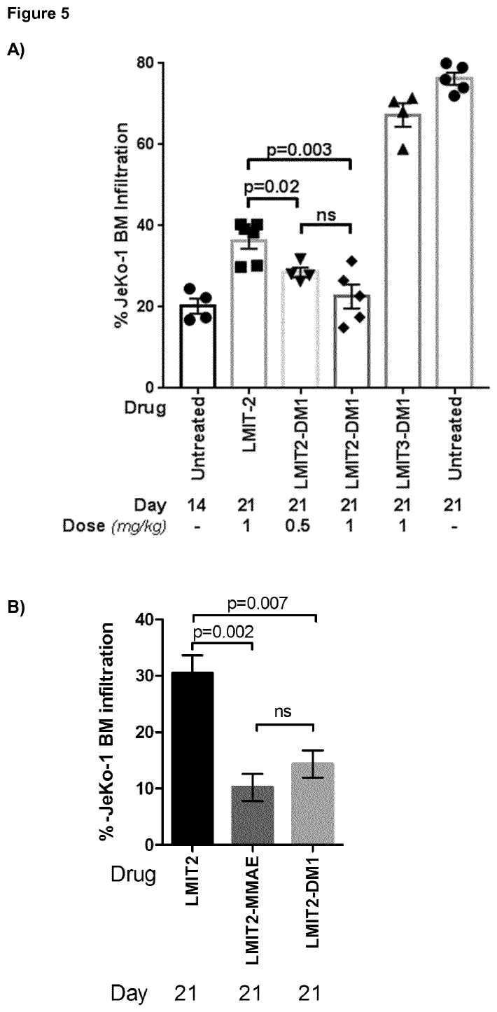

Although it has been disclosed that the microtubule-targeting drug paclitaxel, which kills cells by modulating microtubule dynamics, has a strong synergy with known immunotoxins, other modulators of microtubule dynamics like vincristine show less effect in this respect (Hollevoet et al. 2014, Mol Cancer Ther; 13(8):2040-9). Accordingly, successful therapeutic combination of immunotoxins and microtubule modulating drug may not always work, i.e. lead to the intended effect. However, the inventors of the present invention discovered that mertansine and monomethyl auristatin which bind a novel microtubule binding site, distinct from paclitaxel, can reproduce the strong paclitaxel-like synergy when linked to the immunotoxins of the present invention. In fact, the conjugation of mertansine DM1 to the immunotoxins of the present invention reproduces similar activity than the addition of paclitaxel as a second drug to immunotoxins, thereby demonstrating that it is possible to deliver the two payloads directly to the tumor cell. The maximally tolerated dose of conventional immunotoxins and Duotoxins as used in FIG. 5 are the same. Thus, unspecific side effects likely are similar for the two drugs. In line, the Duotoxins of the present invention increase efficacy when compared to immunotoxin without increasing side effects.

Compared with commonly used whole antibodies for the construction of ADCs, the molecules disclosed here are generally smaller and have only one antigen binding site, resulting in two major advantages. First, because the cross-linking of target receptors with bivalent antibodies changes internalization kinetics of the target protein, the monovalent antibody format shows favorable activity over bivalent ADCs. Second, while the half-life of whole antibody ADCs is more than one week, the smaller drugs disclosed herein have a half-life of less than one hour. This facilitates the control of side effects in patients.

Thus, in a general aspect, the present invention provides for a conjugate comprising an immunotoxin linked to a therapeutic agent, wherein said immunotoxin comprises a binding domain fused to an adenosine diphosphate (ADP) ribosylating toxin. The term “conjugate” means that two therapeutic agents are associated on one immunoglobulin, wherein said conjugate benefits from the simultaneous therapeutic impact of both entities. In this respect, said new immunotoxin conjugates comprise an adenosine diphosphate (ADP) ribosylating toxin and a therapeutic small molecule combined on one antibody molecule, thereby allowing for the delivery of the two synergistically acting drugs to a target cell without loss of activity.

The term “immunotoxin” as used herein refers to human-made immuno-conjugates that comprise a proteinaceous binding domain linked to a toxin, preferably an adenosine diphosphate (ADP) ribosylating toxin. When said proteinaceous binding domain binds to a target cell, the immunotoxins are taken in through endocytosis, and the toxin kills the cell. The term “binding domain” as used herein can be interchangeably used with the terms “targeting portion” and refers to a peptide or polypeptide capable of binding to a target structure. The term “capable of binding” can be understood as the capability of said protein to bind to target cells, both in vitro and in vivo. The “target cell” is the cell to which the “binding domain” or “targeting portion” of the present invention binds to.

Thus, when bringing the target cell in contact with the “binding domain”, the “binding domain” is capable of specifically binding to a surface molecule or structure on said “target cell”. “Specifically binding” as used in this regard refers to the binding affinity of the binding domain or targeting portion to the surface molecule on the target cell. Preferably, the surface molecule on the target cell is a cell surface protein which it is desired to detect. Cell surface proteins” in the context of the present invention are proteins that are embedded in or span the layer of cell membranes of more complex organisms. These proteins are integral to the way in which a cell interacts with the environment around it, including other cells. Some of these proteins, especially ones that are exposed to the external side of the membrane, are called glycoproteins because they have carbohydrates attached to their outer surfaces. Said cell surface protein are selected from transport proteins allowing solutes to flow into or out of the cell, recognition proteins, which form contacts between adjoining cells in order to facilitate cell to cell communications through which signals can flow, receptor proteins allowing communication with substances that serve as signaling molecules such as hormones, and enzyme catalyzing reactions related directly to the cell membrane. The cell surface proteins the conjugates of the present invention target are preferably molecules on the target cells which allow to selectively targeting said cells. Targeting said cell surface proteins by the conjugates described herein allows for the treatment of cell-based diseases described elsewhere herein.

Preferably, the binding domain comprised by the immunotoxin as described herein is capable of binding to cell surface proteins of various types of cells of the organism, such as epithelial cell, neuronal cells, blood cells, tissue cells, muscle cells, fibroblasts, germ cells or immune cells including dendritic cells, T-cells, monocytes, B-cells, or precursors, just to name some. Said cells are the respective target cells described elsewhere herein. In particular, said cell surface proteins are surface structures expressed on pathological cells, such as cancer cells or cells involved in abnormal immune response like autoimmune diseases. The conjugates of the present invention comprising the binding domain described herein are capable of binding to the respective cell surface protein on the target cells which induces endocytosis of the drug, thereby unfolding the synergistic effect of the toxin and the therapeutic drug described elsewhere herein. Thus, when targeting cell surface proteins expressed on cancer cells or cells involved in autoimmune diseases, the conjugates described herein are directly carried to the place of the disease, thereby facilitating treatment of the respective syndrome.

When targeting cancer cells, the binding domain is capable of binding to so called tumor antigens on the targeted cancer cell. The term “tumor antigen” as used herein may be understood as those antigens that are presented on tumor cells. These antigens can be presented on the cell surface with an extracellular part, which is often combined with a transmembrane and cytoplasmic part of the molecule. These antigens can sometimes be presented only by tumor cells and never by the normal ones. Tumor antigens can be exclusively expressed on tumor cells or might represent a tumor specific mutation compared to normal cells. In this case, they are called tumor-specific antigens. More common are antigens that are presented by tumor cells and normal cells, and they are called tumor-associated antigens. These tumor-associated antigens can be overexpressed compared to normal cells or are accessible for antibody binding in tumor cells due to the less compact structure of the tumor tissue compared to normal tissue. Non-limiting examples of tumor antigens are EGFR, EGFRvIII, MCSP, EpCAM, Carbonic anhydrase IX (CAIX), fms like tyrosine kinase (FLT-3), CD22, CD30, CD33, CD138, Her2/neu, CD44v6, mesotheline (MSLN) and Muc-1 (Liu, Br. J. Cancer 82/12 (2000), 1991-1999; Bonner, Semin. Radiat. Oncol. 12 (2002), 11-20; Kiyota, Oncology 63/1 (2002), 92-98; Kuan, Brain Tumor Pathol. 17/2 (2000), 71-78), EGFRvIII (Kuan, Brain Tumor Pathol. 17/2 (2000), 71-78), Carboanhydrase IX (MN/CA IX) (Uemura, Br. J. Cancer 81/4 (1999), 741-746; Longcaster, Cancer Res. 61/17 (2001), 6394-6399; Chia, J. Clin. Oncol. 19/16 (2001), 3660-3668; Beasley, Cancer Res. 61/13 (2001), 5262-5267), CD33 (Abutalib, Curr Pharm Biotechnol. 7 (2006), 343-69), MCSP (Campoli, Crit Rev Immunol. 24 (2004), 267-96), or IgE (Infuhr, Allergy 60 (2005), 977-85). According to a preferred embodiment, it is envisaged that the binding domain described herein is capable of binding to the cell surface proteins CD22, mesotheline (MSNL), fms like tyrosine kinase 3 (FLT-3), HER2/neu or CD138. However, other cell surface proteins can be equally bond by the binding domain comprised by Duotoxins of the invention and the skilled person is well aware how to construct the respective binding domains.

The affinity of the binding domain of the conjugate of the present invention to a cell surface protein can be defined in terms of a dissociation constant (KD). The binding domain or targeting protein specifically binds a cell surface protein when the KD is less than about 1×10⁻⁷ M, such as about 1×10⁻⁸ or less, such as about 1×10⁻⁹ M or less, about 1×10⁻¹⁰ M or less, about 1×10⁻¹¹ M or less, about 1×10⁻¹² M or even less, and binds to the predetermined cell surface protein with an affinity corresponding to a KD that is at least ten-fold lower than its affinity for binding to a non-specific antigen (such as BSA), such as at least 100 fold lower, for instance at least 1,000 fold lower, such as at least 10,000 fold lower. The term “KD” refers to the dissociation constant of a particular protein-protein binding interaction as is known in the art.

The term “binding domain” according to the present invention refers to an immunoglobulin or a proteinaceous binding partner. Said binding domain can be chemically modified to increase its stability. Chemically modified polypeptides or peptide analogs include any functional chemical equivalent of the polypeptide characterized by its increased stability and/or efficacy in vivo and in vitro. The term “peptide analog” also refers to any amino acid derivatives. A peptide analog can be produced by procedures that include, but are not limited to, modifications to side chains.

In view of the present invention, the binding domain comprised by the conjugate described herein is preferably an antibody or a functional fragment thereof. An antibody or functional fragment of the present invention is hence capable of binding to a cell surface protein, i.e. to a specific antigen or epitope. Thus, an antibody construct according to the invention comprises the minimum structural requirements of an antibody which allow for the epitope binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region).

The antibodies on which the conjugates according to the invention are based may include for example monoclonal, recombinant, chimeric, deimmunized, humanized and human antibodies or functional fragments thereof. Within the definition of “antibody” according to the invention are full-length or whole antibodies including camelid antibodies and other immunoglobulin antibodies generated by biotechnological or protein engineering methods or processes. These full-length antibodies may be for example monoclonal, recombinant, chimeric, deimmunized, humanized and human antibodies.

According to a preferred embodiment, the binding domain comprised by the conjugate of the present invention is a functional fragment of an antibody, preferably a monovalent antibody fragment. Such fragments are fragments of full-length antibodies described elsewhere herein and comprise VH, VHH, VL, (s)dAb, Fv, Fd, Fab, Fab′, F(ab′)2 or “r IgG” (“half antibody”). Preferred antibody fragment are selected from the group consisting of an antibody binding fragment Fab, a disulphide-stabilized Fv (dsFv), or a single-chain Fv (scFv).

It is particularly preferred that the binding domain comprised by the immunotoxin described elsewhere herein comprises variable regions. The variable regions, i.e. the variable light chain (“L” or “VL”) and the variable heavy chain (“H” or “VH”) are understood in the art to provide the binding domain of an antibody. This variable regions harbor the complementary determining regions. The term “complementary determining region” (CDR) is well known in the art to dictate the antigen specificity of an antibody. The term “CDR-L” or “L CDR” refers to CDRs in the VL, whereas the term “CDR-H” or “H CDR” refers to the CDRs in the VH. Preferably, the binding domain comprises a CDR-L1, CDR-L2 and CDR-L3 and/or a VH region comprising CDR-H1, CDR-H2 and CDR-H3 selected from:
(a) CDR-L1 as shown in SEQ ID NO: 1, CDR-L2 as shown in SEQ ID NO: 2 and CDR-L3 as shown in SEQ ID NO: 3 and/or CDR-H1 as shown in SEQ ID NO: 4, CDR-H2 as shown in SEQ ID NO: 5 and CDR-H3 as shown in SEQ ID NO: 6;
(b) CDR-L1 as shown in SEQ ID NO: 7, CDR-L2 as shown in SEQ ID NO: 8 and CDR-L3 as shown in SEQ ID NO: 9 and/or CDR-H1 as shown in SEQ ID NO: 10, CDR-H2 as shown in SEQ ID NO: 11 and CDR-H3 as shown in SEQ ID NO: 12;
(c) CDR-L1 as shown in SEQ ID NO: 13, CDR-L2 as shown in SEQ ID NO: 14 and CDR-L3 as shown in SEQ ID NO: 15 and/or CDR-H1 as shown in SEQ ID NO: 16, CDR-H2 as shown in SEQ ID NO: 17 and CDR-H3 as shown in SEQ ID NO: 18; and
(d) CDR-L1 as shown in SEQ ID NO: 19, CDR-L2 as shown in SEQ ID NO: 20 and CDR-L3 as shown in SEQ ID NO: 21 and/or CDR-H1 as shown in SEQ ID NO: 22, CDR-H2 as shown in SEQ ID NO: 23 and CDR-H3 as shown in SEQ ID NO: 24.

More preferably, said binding domain comprises a VL region and/or a VH region selected from:
(a) a VL region as shown in SEQ ID NO: 25 and/or a VH region as shown in SEQ ID NO: 26;
(b) a VL region as shown in SEQ ID NO: 27 and/or a VH region as shown in SEQ ID NO: 28;
(c) a VL region as shown in SEQ ID NO: 29 and/or a VH region as shown in SEQ ID NO: 30; and
(d) a VL region as shown in SEQ ID NO: 31 and/or a VH region as shown in SEQ ID NO: 32.

According to a preferred embodiment of the binding domain of the invention, the pairs of VH-regions and VL-regions are in the format of a single chain antibody (scFv). The VH and VL regions are arranged in the order VH-VL or VL-VH. It is preferred that the VH-region is positioned N-terminally to a linker sequence. The VL-region is positioned C-terminally of the linker sequence. A preferred embodiment of the above described polypeptide of the invention is characterized by a binding domain comprising an amino acid sequence selected from the group consisting of SEQ ID NOs: 40 and 41, SEQ ID NOs: 43 and 44; SEQ ID NOs: 45 and 46, or SEQ ID NOs: 47 and 48.

Binding domains according to the invention may also be modified fragments of antibodies, also called antibody variants, such as scFv, scFab, Fab2, Fab3, diabodies, single chain diabodies, “minibodies” exemplified by a structure which is as follows: (VH-VL-CH3)2, (scFv-CH3)2 or (scFv-CH3-scFv)2, and single domain antibodies such as nanobodies or single variable domain antibodies comprising merely one variable domain, which might be VHH, VH or VL, that specifically bind an antigen or epitope independently of other V regions or domains.

In some embodiments an antibody is an aptamer, including a Spiegelmer®, described in e.g. WO01/92655. An aptamer is typically a nucleic acid molecule that can be selected from a random nucleic acid pool based on its ability to bind a selected other molecule such as a peptide, a protein, a nucleic acid molecule a or a cell. Aptamers, including Spiegelmers, are able to bind molecules such as peptides, proteins and low molecular weight compounds. Spiegelmers® are composed of L-isomers of natural oligonucleotides. Aptamers are engineered through repeated rounds of in vitro selection or through the SELEX (systematic evolution of ligands by exponential enrichment) technology. The affinity of Spiegelmers to their target molecules often lies in the pico- to nanomolar range and is thus comparable to immunoglobulins. An aptamer may also be a peptide. A peptide aptamer consists of a short variable peptide domain, attached at both ends to a protein scaffold.

The antibody or functional fragment thereof according to the present invention may be monoclonal or polyclonal. The term “polyclonal” refers to immunoglobulins that are heterogenous populations of immunoglobulin molecules derived from the sera of animals immunized with an antigen or an antigenic functional derivative thereof. For the production of polyclonal immunoglobulins, one or more of various host animals may be immunized by injection with the antigen. Various adjuvants may be used to increase the immunological response, depending on the host species. “Monoclonal immunoglobulins” or “Monoclonal antibodies” are substantially homogenous populations of immunoglobulins to a particular antigen. They may be obtained by any technique which provides for the production of immunoglobulin molecules by continuous cell lines in culture. Monoclonal immunoglobulins may be obtained by methods well known to those skilled in the art (see for example, Köhler et al., Nature (1975) 256, 495-497, and U.S. Pat. No. 4,376,110). Routine methods known to those skilled in the art enable production of both immunoglobulins and immunoglobulin fragments in both prokaryotic and eukaryotic organisms. In more detail, an immunoglobulin may be isolated by comparing its binding affinity to a protein of interest with its binding affinity to other polypeptides. Humanized forms of the antibodies may be generated using one of the procedures known in the art such as chimerization or CDR grafting.

In general, techniques for preparing monoclonal antibodies and hybridomas are well known in the art. Any animal such as a goat, a mouse or a rabbit that is known to produce antibodies can be immunized with the selected polypeptide. In a preferred embodiment, the immunoglobulin of the present invention is obtained by immunizing a rodent, preferably a rat, by immunization with a cell surface peptide. Methods for immunization are well known in the art. Such methods include subcutaneous or intraperitoneal injection of the polypeptide. One skilled in the art will recognize that the amount of polypeptide used for immunization and the immunization regimen will vary based on the animal which is immunized, including the species of mammal immunized, its immune status and the body weight of the mammal, as well as the antigenicity of the polypeptide and the site of injection. The polypeptide may be further modified or administered in an adjuvant in order to increase the peptide antigenicity. Methods of increasing the antigenicity of a polypeptide are well known in the art. Such procedures include coupling the antigen with a heterologous protein (such as globulin or b-galactosidase) or through the inclusion of an adjuvant during immunization. Typically, the immunized mammals are bled and the serum from each blood sample is assayed for particular antibodies using appropriate screening assays known to those skilled in the art.

For monoclonal immunoglobulins, lymphocytes, typically splenocytes, from the immunized animals are removed, fused with an immortal cell line, typically myeloma cells, such as SP2/0-Agl4 myeloma cells, and allowed to become monoclonal immunoglobulin producing hybridoma cells. Typically, the immortal cell line such as a myeloma cell line is derived from the same mammalian species as the lymphocytes. Illustrative immortal cell lines are mouse myeloma cell lines that are sensitive to culture medium containing hypoxanthine, aminopterin and thymidine (“HAT medium”). Typically, HAT-sensitive mouse myeloma cells are fused to mouse splenocytes using 1500 molecular weight polyethylene glycol (“PEG 1500”). Hybridoma cells resulting from the fusion may then be selected using HAT medium, which kills unfused and unproductively fused myeloma cells (unfused splenocytes die after several days because they are not transformed).

Any one of a number of methods well known in the art can be used to identify a hybridoma cell which produces an immunoglobulin with the desired characteristics. Typically the culture supernatants of the hybridoma cells are screened for immunoglobulins against the antigen. Suitable methods include, but are not limited to, screening the hybridomas with an ELISA assay, Western blot analysis, or radioimmunoassay (Lutz et al., Exp. Cell Res. 175, 109-124). Hybridomas prepared to produce the immunoglobulins according to the present invention may for instance be screened by testing the hybridoma culture supernatant for secreted antibodies having the ability to bind to the respective cell surface protein. To produce antibody homologs which are within the scope of the invention, hybridoma cells that tested positive in such screening assays can be cultured in a nutrient medium under conditions and for a time sufficient to allow the hybridoma cells to secrete the monoclonal immunoglobulins into the culture medium. Tissue culture techniques and culture media suitable for hybridoma cells are well known in the art. The conditioned hybridoma culture supernatant may be collected and the immunoglobulins optionally further purified by well-known methods. Alternatively, the desired immunoglobulins may be produced by injecting the hybridoma cells into the peritoneal cavity of an unimmunized mouse. The hybridoma cells proliferate in the peritoneal cavity, secreting the immunoglobulin which accumulates as ascites fluid. The immunoglobulin may be harvested by withdrawing the ascites fluid from the peritoneal cavity with a syringe.

Hybridomas secreting the desired immunoglobulins are cloned and the class and subclass are determined using procedures known in the art. For polyclonal immunoglobulins, immunoglobulin containing antisera is isolated from the immunized animal and is screened for the presence of immunoglobulins with the desired specificity using one of the above-described procedures. The above-described antibodies may also be immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art.

A plurality of conventional display technologies is available to select an immunoglobulin or immunoglobulin fragments. Li et al. (Organic & Biomolecular Chemistry (2006), 4, 3420-3426) have for example demonstrated how a single-chain Fv fragment capable of forming a complex with a selected DNA adapter can be obtained using phage display. Display techniques for instance allow the generation of engineered immunoglobulins and ligands with high affinities for a selected target molecule. It is thus also possible to display an array of peptides or proteins that differ only slightly, typically by way of genetic engineering. Thereby it is possible to screen and subsequently evolve proteins or peptides in terms of properties of interaction and biophysical parameters. Iterative rounds of mutation and selection can be applied on an in vitro basis.

According to the present invention, the immunotoxin described herein comprises a binding domain fused to an adenosine diphosphate (ADP) ribosylating toxin. The term “adenosine diphosphate (ADP) ribosylating toxin” as used herein refers to bacterial or plant toxins which covalently transfer an ADP-ribose moiety of NAD+ to target proteins of infected eukaryotes, to yield nicotinamide and a free hydrogen ion. Said ADP-ribosylating toxins produced in nature as enzyme precursors, consisting of a binding domain, a catalytic active domain responsible for ADP-ribosylation and a domain for translocation of the enzyme across the membrane of the cell. These domains can exist in three forms: first, as single polypeptide chains with all domains covalently linked; second, in multi-protein complexes with the domains bound by non-covalent interactions; and, third, in multi-protein complexes with the domains not directly interacting, prior to processing (Krueger & Barbieri, Clinical Microbiology Reviews 1995; 8 (1): 34-47). Upon activation, these toxins ADP-ribosylate any number of eukaryotic proteins. Such mechanism is crucial to the instigation of the diseased states associated with ADP-ribosylation. GTP-binding proteins, in particular, are well-established in the pathophysiology of ADP ribosylating toxins. According to the present invention, the ADP ribosylating toxin is preferably Pseudomonas exotoxin (PE) A or diphtheria toxin.

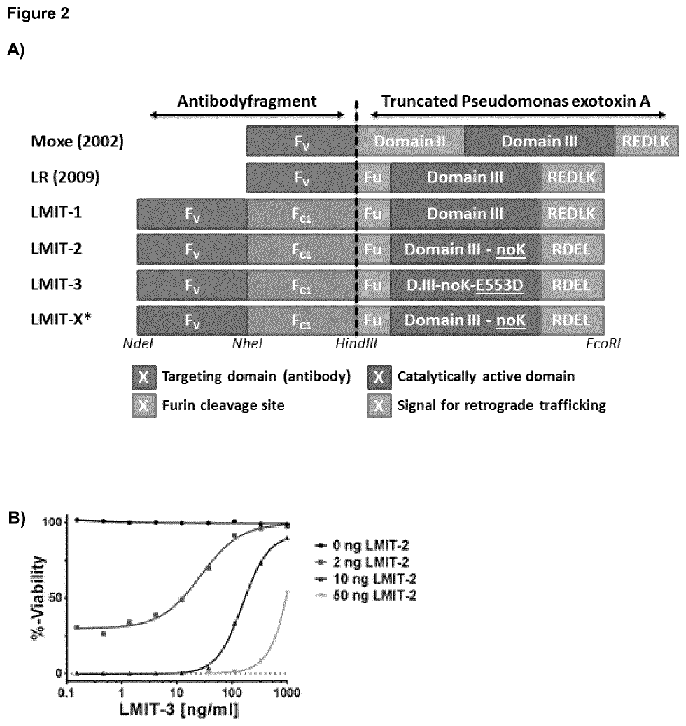

The chemical conjugation of a small molecule to immunotoxins is supposed to reduce the activity of the comprised toxin. However, to enable a conjugation that maintains full immunotoxin activity, some modifications have to be combined resulting in novel immunotoxins according to the present invention. As shown exemplarily for Pseudomonas exotoxin (PE) A, said novel toxins are characterized in (i) lack of a translocation domain, i.e. lack of domain II of Pseudomonas exotoxin (PE) A, but maintenance of the furin cleavage site, and (ii) a catalytic active domain, wherein all lysine residues have been substituted by any other amino acid, i.e. two mutations in domain domain III of Pseudomonas exotoxin (PE) A. Thus, in a general aspect of the invention, the ADP-ribosylating toxin comprises from the amino terminus a furin cleavage site and a catalytic active domain. The modified PE is further characterized by (iii) an unusual C-terminal KDEL-motif “RDEL” (FIG. 2A). Whether a molecule having two or all three changes mentioned herein above simultaneously remains active, has never been tested before. As described in context of the present invention, removing all lysine residues from the ADP-ribosylating toxin allows for an amid-directed coupling of a microtubule targeting therapeutic agent such as mertansine or monomethyl auristatin (MMA) to the immunotoxin of the present invention, without reducing the activity of the coupled toxin.

Accordingly, the ADP-ribosylating toxins of the present invention preferably comprises at the amino terminus the furin cleavage site as shown in SEQ ID NO: 33 or an amino acid sequence having at least 85%, such as 86, 87, 88, 98, 90, 91, 92, 93, 94, 95, 96, 97, 98, 99% identity to the amino acid sequence as shown in SEQ ID NO: 33 and the amino acid in position 6 of the variant of SEQ ID NO: 33 is an arginine. Accordingly, also variants of the furin cleavage site are within the scope of the present invention, wherein the arginine in position 6 of SEQ ID NO: 33 is a key amino acid and necessarily comprised. However, the skilled person is also aware of other furin cleavage sites which can be equally used for the ADP-ribosylating toxins of the present invention. Furin is an enzyme which belongs to the subtilisin-like proprotein convertase family. The members of this family are proprotein convertases that process latent precursor proteins into their biologically active products. Furin is also known as PACE (Paired basic Amino acid Cleaving Enzyme). Accordingly, the furin cleavage site comprised by the immunotoxins described herein is essential for the toxin-activation by furin enzymes and the intended biological effect of the toxins. ADP-ribosylating toxins like PE comprise a natural furin cleavage site (SEQ ID NO: 33), but variants thereof or optimized furin cleavage sites as described for example in Weldon et al. 2015, Bioconjugate Chem.; 26 (6):1120-1128 can also be used within the context of the present invention.

The ADP-ribosylating toxin further comprises from the amino terminus the catalytic active domain of said toxin, wherein all lysine residues have to be substituted by any other amino acid. The term “catalytic active” as used in this regard means that said domain is capable of covalently transferring the ADP-ribose moiety of NAD+ to a target protein as described elsewhere herein. The term “amino acid” refers to any other naturally occurring and non-natural amino acid except lysine, as well as amino acid analogs and amino acid mimetics that function in a manner similar to the naturally occurring amino acids. Said naturally occurring amino acids are alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine and selenocysteine. Amino acid analogs refers to compounds that have the same basic chemical structure as said naturally occurring amino acids, by way of example only, an alpha-carbon that is bound to a hydrogen, a carboxyl group, an amino group, and an R group. Such analogs may have modified R groups (by way of example, norleucine) or may have modified peptide backbones, while still retaining the same basic chemical structure as a naturally occurring amino acid. Non-limiting examples of amino acid analogs include homoserine, norleucine, methionine sulfoxide, and methionine methyl sulfonium. The catalytic domain of the ADP-ribosylating toxin described herein preferably comprises an amino acid sequence as shown in SEQ ID NO: 34 or 35 or an amino acid sequence having at least 85%, such as 86, 87, 88, 98, 90, 91, 92, 93, 94, 95, 96, 97, 98, 99% identity to the amino acid sequence as shown in SEQ ID NO: 34 or 35, wherein the lysine residues have been substituted by any other amino acid. Accordingly, also variants of the catalytic active toxin domain are within the scope of the present invention.

The term “variant” as used herein in view of the ADP-ribosylating toxin refers to a peptide that differs from the natural sequence, i.e. the natural furin cleavage site or the natural catalytic active toxin domain, but retains essential properties. Generally, differences are limited so that the sequences of the reference peptide and the variant are closely similar overall and, in many regions, identical. A variant and the natural sequence may differ in amino acid sequence by one or more modifications (e.g., substitutions, additions, and/or deletions). A substituted or inserted amino acid residue may or may not be one encoded by the genetic code. A variant of a peptide may be naturally occurring, such as an allelic variant, or it may be a variant that is not known to occur naturally.

As used herein, the term “% identity” refers to the percentage of identical amino acid residues at the corresponding position within the sequence when comparing two amino acid sequences with an optimal sequence alignment as exemplified by the ClustalW or X techniques as available from www.clustal.org, or equivalent techniques. For example, in case of alignments of the comprised natural toxin sequences, each of the sequences shown in SEQ ID NOs: 34 and 35 serves as reference sequence and is aligned with a toxin sequences comprising a certain sequence variability. Accordingly, both sequences (natural sequence and sequence with variability) are aligned, identical amino acid residues between both sequences are identified and the total number of identical amino acids is divided by the total number of amino acids (amino acid length) of SEQ ID NO: 34 and 35. The result of this division is a percent value, i.e. percent identity value/degree.

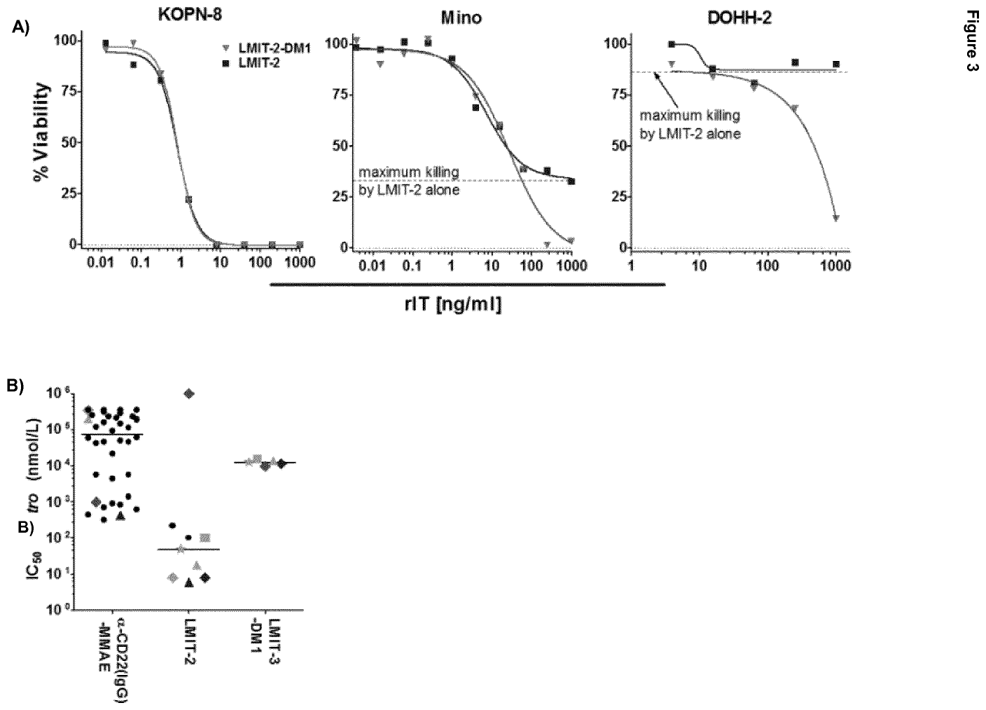

The ADP-ribosylating PE toxin further comprises at the carboxy terminus a modified REDLK motif, i.e. an amino acid sequence as shown in SEQ ID NO: 36. Preferably, the ADP-ribosylating PE toxin comprises at the carboxy terminus the amino acid sequence RDEL (SEQ ID NO: 37). As shown in FIGS. 2 and 3, the modified ADP-ribosylating toxins of the present invention still exhibit the same or similar activity than the parental, unmodified toxins, although comprising several modifications. Functionality of the immunotoxins comprised by the present invention can be determined using the methods described in the Examples. To provide sufficient amino acid side chains suitable for the conjugation reaction to the therapeutic drug, additional to the Fv region the first constant region Fc₁ of the immunoglobulin has to be added, thereby changing the antibody format from a dsFv to an Fab (see FIG. 2A). Preferably, the ADP-ribosylating toxin according to the present invention is Pseudomonas exotoxin (PE) A. However, other bacterial toxins such as diphtheria toxin which also comprise a catalytic active domain fusible to an immunoglobulin are equally suitable and within the scope of the present invention. Preferably, the ADP-ribosylating toxin used according to the present invention comprises an amino acid sequence as shown in SEQ ID NO: 38.

The novel conjugates described herein comprise an immunotoxin comprising a binding domain fused to ADP-ribosylating toxin. The term “fused” as used in this context refers to the covalently joining together of two heterogeneous polypeptides of different origins, including chemical conjugation or recombinant means. For example, as shown in FIG. 2A of the present invention, a nucleic acid encoding for the respective binding domain and a nucleic acid encoding for the ADP ribosylating toxin can be operably linked to a coding sequence and transcribed. “Operably linked” means that the DNA sequences being linked are contiguous, and in reading phase or in-frame. Thus, the resulting recombinant fusion protein is a single protein comprising two polypeptide segments that correspond to the binding domain polypeptide and the toxin polypeptide, which segments are not normally so joined in nature. Accordingly, in the present invention, the term “fusion protein” refers to a polypeptide in which the binding domain and an adenosine diphosphate (ADP) ribosylating toxin are covalently combined to each other.

The modified immunotoxins described herein are further linked to a small molecule therapeutic drug, a microtubule-targeting therapeutic agent to form the novel “Duotoxins” according to the present invention, which allow for a simultaneous delivery of two synergistically acting drugs to target cells. The term “therapeutic agent” or “drug-like molecule” as used in this regard means any compound that has characteristics that make it suitable for use in medicine and, thus, is useful for therapeutic purposes. Thus, for example and without limitation, a therapeutic agent is a molecule that is synthesized by the techniques of organic chemistry, or by techniques of molecular biology or biochemistry, and is in some aspects a small molecule as defined herein. A drug-like molecule, in various aspects, additionally exhibits features of selective interaction with a particular protein or proteins and is bioavailable and/or able to penetrate cellular membranes either alone or in form of the conjugates of the present invention. Therapeutic agents are further understood to mean any compound that can be administered to a patient for the treatment of a disease or pathological condition. Preferably, said disease or pathological condition is a cell-based disease or condition whereas the disease is caused by the uncontrolled or the over-shooting cell growth or activity of a defined cell population. The term “cell-based” means in this regard that these diseases or conditions directly involve cells of the subject to be treated, preferably cells of the immune system, which can be targeted by the conjugates described herein. In some embodiments the “cell-based disease or condition” is cancer. In some embodiments the “cell-based disease or condition” is an infectious disease. In some embodiments the “cell-based disease or condition” is an autoimmune disease.

As stated above, therapeutic agents useful in this regard include without limitation drug-like molecules, proteins, peptides, and small molecules. Protein therapeutic agents include, without limitation peptides, enzymes, structural proteins, receptors and other cellular or circulating proteins as well as fragments and derivatives thereof. In a preferred embodiment, the therapeutic agent has cytostatic and/or cytotoxic activity. The term “cytotoxic” refers to an agent, which can be administered to kill or eliminate a cancer cell. The term “cytostatic” refers to an agent, which can be administered to restrain tumor proliferation rather than induce cytotoxic cytoreduction yielding an elimination of the cancer cell from the total viable cell population of the patient. Cytotoxic and cytostatic agents have gained wide spread use as chemotherapeutics in the treatment of various cancer types and are well known in the art.

Preferably, the therapeutic agent is an anti-cancer drug or agent. The term “anti-cancer drug” or “anti-cancer agent” used herein refers to a chemotherapeutic agent used to treat tumors including malignant tumors. Anti-cancer drugs or agents typically refers to compounds which intervenes in various metabolic pathways of cancer cells to exert its anticancer activity by mainly inhibiting the synthesis of nucleic acids of cancer cells. The anti-cancer agent utilizes the difference in susceptibility to drugs between normal cells and cancer cells, while it acts more selectively against cancer cells with relatively less toxicity to normal cells. However, normal cells are also damaged to some degree by the anti-cancer agent, resulting in the presence of adverse side effects. This is because the anti-cancer agent acts on any cell that has a rapid cell division, so it does not only act on the rapidly dividing cancer cells but also the bone marrow, gastrointestinal tract and hair follicular cells, which are also rapidly dividing cells, respectively, are also affected by the anti-cancer agent. The common side effects of these drugs include, are not limited to, temporary reduction of blood cells, nausea, vomiting, diarrhea, loss of appetite, and hair loss. Currently, anti-cancer agents used for cancer treatment are divided into six following categories according to their biochemical functional mechanisms: alkylating agents, metabolic antagonists, antibiotics, mitotic inhibitors, hormones and others. In some embodiments, the anti-cancer agent includes, but is not limited to, cisplatin, doxorubicin, etoposide, paclitaxel, docetaxel, fluoropyrimidine, oxalplatin, campthotecan, Belotecan, podophyllotoxin, vinblastine sulfate, cyclophosphamide, actinomycin, vincristine sulfate, methotrexate, bevacuzumab, thalidomide, eriotinib, gefitinib, camptothecin, Tamoxifen, Anasterozole, Gleevec, 5-fluorouracil (5-FU), Floxuridine, Leuprolide, Flutamide, Zoledronate, Vincristine, Gemcitabine, Streptozocin, Carboplatin, Topotecan, Irinotecan, Vinorelbine, hydroxyurea, Valrubicin, retinoic acid, Meclorethamine, Chlorambucil, Busulfan, Doxifluridine, Vinblastin, Mitomycin, Prednisone, Testosterone, Mitoxantron, aspirin, salicylates, ibuprofen, naproxen, fenoprofen, indomethacin, phenylbutazone, cyclophosphamide, mechlorethamine, dexamethasone, prednisolone, celecoxib, valdecoxib, nimesulide, cortisone, and corticosteroid, while there is no limitation in terms of the type of anti-cancer agent including a chemical compound, a hormone, an antibody and the like.

Therapeutic agents in view of the present invention also include, in various embodiments, radioactive material. In some aspects therapeutic agents include small molecules, i.e., compounds having a molecular weight of less than 1000 Daltons, typically between 300 and 700 Daltons. The term “small molecule,” as used herein, refers to a chemical compound, for instance a peptidometic that may optionally be derivatized, or any other low molecular weight organic compound, either natural or synthetic. Such small molecules may be a therapeutically deliverable substance or may be further derivatized to facilitate delivery. By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, about 1000 or more Daltons.

According to a preferred embodiment, the anti-cancer drug comprised by the conjugates of the present invention is a microtubule-targeting drug. The term “microtubule-targeting drug” refers to anti-cancer drugs or agents altering or disrupting microtubule organization or microtubule dynamics, preferably leading to mitotic arrest and cell death. Microtubules are central to intracellular transport and are the main component of the spindle complex responsible for chromosome separation during cell division. They are composed of α- and β-tubulin heterodimers. The dynamic of microtubules is tightly regulated to guaranty a high level of plasticity and ultimately cell survival. Changes in microtubule length are achieved by disassembling the “minus” end or by extending the “plus” end. A variety of substances, including paclitaxel or vincristine, alter microtubule dynamics (Jordan & Wilson 2004, Nat. Rev. Cancer; 4:253-265; Stanton et al. 2011, Med. Res. Rev.; 31:443-481). Therefore, microtubules represent a validated primary oncologic target of microtubule-targeting agents in humans. These drugs are sub-divided into microtubule stabilizers and microtubule destabilizers, whereas drugs with the same binding site commonly have similar effects on microtubule dynamics. Taxanes like the paclitaxel bind to a unique site at the inner wall of microtubules. Vincristine-type drugs bind to a binding groove on the outside. Intriguingly, tumor entities are often exclusively sensitive to defined microtubule modifiers. B-cell malignancies are very sensitive to the vincristine type, but not to paclitaxel, while many solid tumors are sensitive to paclitaxel but not to vincristine (Jordan & Wilson 2004, Nat. Rev. Cancer; 4:253-265; Stanton et al. 2011, Med. Res. Rev.; 31:443-481).

However, the present invention preferably refers to the novel small molecule of the class of microtubule dynamic modifiers like mertansine and monomethyl auristatin (MMA), which bind the tip of microtubules, suppresses microtubule dynamics and result in mitotic arrest and cell death (Lopus 2011, Cancer Letter; 307:113-118). Mertansines by themselves are highly toxic to humans, and only with the development of serum stable linkers the administration linked to antibodies has become safe (Lewis Philips 2008, Cancer Res.; 68:92809290). Thus, when combined with immunotoxins, the novel Duotoxins described herein can selectively target the strong synergistic effects of a microtubule modifier plus immunotoxin to a target cell while healthy cells are not affected from both, the toxin and the therapeutic agent. In a preferred embodiment, the microtubule-targeting drug is selected from mertansine and monomethyl auristatin (MMA). Said mertansine is preferably myatansinoid (DM1), which has been successfully linked to the immunotoxins of the present invention and exhibits a strong synergistic effect in combination with said immunotoxin both in vitro and in vivo (see FIGS. 3 and 5). Alternatively, the conjugates described herein may comprise monomethyl auristatin (MMA), such as monomethyl auristatin E (MMAE) or monomethyl auristatin D (MMAD), equally resulting in the desired synergistic effect.

According to the present invention, the conjugates described herein comprise an immunotoxin linked to a therapeutic agent. The term “linked” as used in this regard refers to the chemical conjugation of the immunotoxin described elsewhere herein to the therapeutic agent described elsewhere herein via a suitable linker. To chemically link a small molecule therapeutic drug like DM1 to a protein, distinct linker chemistries can be used. Linker moieties have a reactive group which defines the target amino acid at the protein. Possible reactive targets are the amide side chain of lysines, the thiol side chain of cysteines, or the carboxyl side chain of glutamate or aspartate. The linkers that attack the carboxyl group are not stable in the serum and are therefore not useful for the conjugation of the present invention. The steps needed to link for example DM1 via cysteines are chemically more complex and need several levels of controls (Junutula et al. 2008, Nat. Biotechnol.; 26:925-932). Therefore, the easiest and the most stable linking chemistry for DM1 is currently the amid-directed coupling via lysine residues (Lewis et al. 2008, Cancer Res.; 68:9280-9290; Doronina et al. 2003, Nat. Biotechnol.; 21:778-784). However, when fluorescent Alexa molecules (Invitrogen) have been linked to immunotoxins using amid-directed reactions, said immunotoxins carrying lysine-linked Alexa molecules are surprisingly three to ten-fold less active (unpublished data). Accordingly, the amid-directed coupling possibly disrupts the activity of the immunotoxin sterically. Therefore, according to the present invention, immunotoxins have been cloned which lacks all lysines residues as exemplary shown for Pseudomonas exotoxin (PE) A (FIG. 2A). As described herein above, the lysine-free PE was achieved by (i) deleting domain II, by introducing the two mutations K590R and K606R into domain III, and by exchanging the carboxy terminal REDLK with RDEL. Whether a molecule that has all three changes simultaneously remains active has not been tested before, using the test described in the Examples.

In addition, to ensure enough lysine residues for a successful coupling reaction, the constant region of the human IgG has been added to the antibody moiety (FIG. 2A). Additionally, a novel restriction site between the Fv and the Fc were introduced to facilitate future exchange of antibodies by “cut and paste” cloning. The final molecule (LIMIT-2) which consists of a Fab and a toxin without lysine residues could be successfully used for amid-coupling of DM1 (LIMIT-2-DM1). The light chain variable and constant domain full sequence as comprised by LMIT-2 is shown in SEQ ID NO: 40 and the heavy chain variable and constant domain full sequence as comprised by LMIT-2 is shown in SEQ ID NO: 41. The complete LMIT-2 heavy chain full sequence comprising the peptide linker of SEQ ID NO: 49, the furin cleavage site of SEQ ID NO: 33, the catalytic active domain of SEQ ID NO: 34 and the amino acid sequence of SEQ ID NO: 37 is shown in SEQ ID NO: 42. As an essential control, an identical constructed (LMIT-3) comprising a mutation at position 553 of PE (glutamate mutated to aspartate, E553D) has been produced. The E553D mutation completely disrupts the ADP-ribosylation, i.e. LMIT-3 is inactive by itself but can block the cytotoxic effects of LMIT-2 in a dose dependent manner (FIG. 2B).

Alternatively to the light chain variable and constant domain full sequence of SEQ ID NO: 40 and the heavy chain variable and constant domain full sequence of SEQ ID NO: 41 that may be comprised by LMIT-2 as shown SEQ ID NO: 42, the Duotoxin conjugates of the present invention may comprise the light chain variable and constant domain full sequence as shown in SEQ ID NO: 43 and the heavy chain variable and constant domain full sequence as shown in SEQ ID NO: 44, the light chain variable and constant domain full sequence as shown in SEQ ID NO: 45 and the heavy chain variable and constant domain full sequence as shown in SEQ ID NO: 46, or the light chain variable and constant domain full sequence as shown in SEQ ID NO: 47 and the heavy chain variable and constant domain full sequence as shown in SEQ ID NO: 48. However, similar Duotoxins comprising binding domains directed against different cell surface molecules can be easily produces by the skilled artisan.

Accordingly, in a preferred embodiment, the linker as used for the conjugates of the present invention is a serum-stable linker. “Serum-stable” means in this regard that conjugates comprising said linker retain their therapeutic activity under neutral pH conditions of human serum, which allows that the conjugates are transported in the organism without cleavage or degradation. Such serum-stable linkers have for example be described in Cazzamalli et al. 2017, J. Control Release (28); 246: 39-45. Such serum-stable linker must further be of sufficient length to allow proper folding of the resulting conjugate. Preferred linkers in this regard are chemical linker, but the skilled person is aware of other serum-stable linkers suitable in this regard. In a preferred embodiment, the therapeutic agent, preferably myatansinoid (DM1) is linked to the immunotoxin by succinimidyl 4-(N-maleimidomethyl)cyclohexane-1-carboxylate (SMCC). In another preferred embodiment, the therapeutic agent, preferably monomethyl auristatin E (MMAE) and monomethyl auristatin D (MMAD) are linked to the immunotoxin by a OSu-Glu-vc-PAB-linker (Ross and Wolfe, Journal of Pharmaceutical Sciences 2016; 105(2): 391-397) or a thiol-linker (Creative® Biolabs, Cat. No: ADCsR-044CL, Product Information). In view of the present invention it is envisaged that at least one molecule of a therapeutic agent, preferably 2, 3, 4, 5, 6, 7, or 8 molecules of a therapeutic agent are linked to the immunotoxin molecule. In a particularly preferred embodiment, 3 or 4 molecules of a therapeutic agent are linked to the immunotoxin. As shown in the Examples of the present invention, in average 3.4 DM1 molecules were coupled pr LIMIT-2 molecule (FIG. 3A). However, the number of molecule of a therapeutic agent comprised by the conjugates described herein much depends on the precise structure of the immunotoxin used in this regard and the selected therapeutic agent. The skilled person aware of said fact and will adapt the conjugates accordingly.

As demonstrated in view of the present invention, the novel Duotoxins described herein show high efficacy under in vitro and in vivo conditions. In this regard Duotoxins were significantly better than either immunotoxins or ADCs (FIGS. 3 and 5), also when combined with a therapeutic agent. Accordingly, the novel conjugates disclosed herein are well suitable for therapeutic purposes and can selectively target the strong synergistic effects of a microtubule modifier and an immunotoxin to a target cell, while healthy cells are not affected from both, the toxin and the therapeutic drug. Accordingly, the present invention also refers to the use of the conjugates described herein in a method of treating a subject suffering from a cell-based disease, preferably suffering from cancer or an autoimmune disease. Equally, the present invention refers to a method for the treatment of a subject suffering from cell-based disease, preferably suffering from cancer or an autoimmune disease, the method comprising administering as therapeutic effective amount of the conjugate of the present invention to the subject in need thereof. Also provided is the use of the conjugate of the present invention for the preparation of a medicament for the treatment of a subject suffering from a cell-based disease, preferably suffering from cancer or an autoimmune disease.

The term “treat”, “treating”, or “treatment” as used herein means to reduce, stabilize, or inhibit the progression of the symptoms associated with the respective disease or disorder. Those in need of treatment include those already with the disease or disorder as well as those prone to having the disease or disorder. Preferably, a treatment reduces, stabilizes, or inhibits progression of a symptom that is associated with the presence and/or progression of a disease or pathological condition. “Treat”, “treating”, or “treatment” refers to both therapeutic treatment and prophylactic or preventative measures, wherein the object is to prevent, slow down (lessen) or at least partially alleviate or abrogate an abnormal, including pathologic, condition in the organism.

The term “subject” as used herein, also addressed as an individual, refers to a mammal. The mammal may be any one of mouse, rat, guineas pig, rabbit, cat, dog, monkey, horse, or human. Accordingly, the mammal of the present invention may be a human or a non-human mammal. Thus, the methods, uses and compositions described in this document are generally applicable to both human and non-human mammals, i.e. to both human and veterinary diseases. Where the subject is a living human who may receive treatment for a disease or condition as described herein, it is also addressed as a “patient”. In some embodiments the subject of the present invention is of a risk to develop a cell-based disease, such as cancer or an autoimmune disease. In some embodiments the subject of the present invention suffers from a cell-based disease, such as cancer or an autoimmune disease. The term “suffering” as used herein means that the subject is not anymore a healthy subject. The term “healthy” means that the respective subject has no obvious or noticeable hallmarks or symptoms of the respective disease or disorder. This further means that the subject suffering from a cell-based disease is a subject “in need” of the respective treatment with said conjugate. Those in need of treatment include those already with the disease or disorder as well as those prone to having the disorder or those in whom the disorder is to be prevented (prophylaxis).

In view of the present invention, the cancer is preferably selected from the group consisting of lymphoma, preferably B-cell malignancies, leukemia, ovarian cancer, breast cancer, lung cancer, prostate cancer, colon cancer, kidney cancer, pancreatic cancer, mesothelioma, lymphoma, liver cancer, urothelial cancer, stomach cancer, and cervical cancer. Preferably, the autoimmune disease is a B-cell mediated disease, preferably any one selected from the group consisting of psoriasis, rheumatoid arthritis, multiple sclerosis, sjogren's syndrome, and guillaume barry syndrome. However, as described elsewhere herein, also other cell-based disease such as inflammatory disorders may be treated wusing the conjugates of the present invention. Typical examples of inflammatory disorder in this context are e.g. acute and chronic inflammatory diseases selected from the group consisting of inflammatory bowel disease, juvenile idiopathic arthritis (JIA), rheumatoid and psoriatic arthritis, seronegative arthritis, or local and systemic inflections, vasculitides, cancer, kidney disease malfunctions, lung injury or pulmonary diseases, allergies, or cardiovascular diseases, just to name some.

The conjugates of the present invention for use in a method of treating a subject suffering from a cell-based disease, such as cancer or an autoimmune disease as described herein are generally administered to the subject in a therapeutically effective amount. Said therapeutically effective amount is sufficient to inhibit or alleviate the symptoms of cell-based disease or disorder. By “therapeutic effect” or “therapeutically effective” is meant that the conjugate for use will elicit the biological or medical response of a tissue, system, animal or human that is being sought by the researcher, veterinarian, medical doctor or other clinician. The term “therapeutically effective” further refers to the inhibition of factors causing or contributing to the disease or disorder. The term “therapeutically effective amount” includes that the amount of the conjugate when administered is sufficient to significantly improve the progression of the disease being treated or to prevent development of said disease. According to a preferred embodiment, the therapeutic effective amount is sufficient to alleviate or heal said cell-based disease, i.e. said cancer or said autoimmune disease.

The therapeutically effective amount will vary depending on the compound, the disease and its severity and on individual factor of the subject such. Therefore, the conjugates of the present invention will not in all cases turn out to be therapeutically effective, because the method disclosed herein cannot provide a 100% safe prediction whether or not a subject may be responsive to said compound, since individual factors are involved as well. It is to expect that age, body weight, general health, sex, diet, drug interaction and the like may have a general influence as to whether or not the compound for use in the treatment of a subject suffering from a respective cell-based disease will be therapeutically effective. Preferably, the therapeutically effective amount of the compound used to treat a subject suffering from the cell-based disease is between about 0.01 mg per kg body weight and about 1 g per kg body weight, such as about 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 100, 200, 300, 400, 500, 600, 700, 800, or about 900 mg per kg body weight. Even more preferably, the therapeutically effective amount of the compound used to treat a subject suffering from the cell-based disease is between about 0.01 mg per kg body weight and about 100 mg per kg body weight, such as between about 0.1 mg per kg body weight and about 10 mg per kg body weight. The therapeutic effective amount of the compound will vary with regard to the weight of active compound contained therein, depending on the species of subject to be treated.

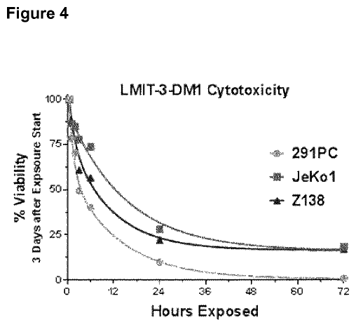

The term “administering” or “administered” means that the conjugates of the present invention are given to the respective subject in an appropriate form and dose and using appropriate measures. The administration of the conjugates according to the present invention can be carried out by any method known in the art. In preferred embodiments, the conjugates are administered intratumorally, parenterally, subcutaneously, intravenously, intramuscularly, intraperitoneally, by intracavitary, intraarterially, transdermally, by application to mucous membranes, intrathecally, intraarticulary, or combinations thereof. Since activity of the Duotoxins described herein depends on the time the cells are exposed and the conjugates need more than 24 hours to reach relevant cytotoxicity, Duotoxins will be more active by continuous administration than by bolus doses (FIG. 4). Accordingly, in view of the present invention, continuous administration of the disclosed conjugates is particularly envisaged. “Continuous” means that the conjugates or permanently administered for a respective time interval without any interruption.

In the scope of the present invention, it is also envisaged that the therapeutic effect is detected by way of surgical resection or biopsy of the affected tissue or organ, which is subsequently analyzed by way of, for example immunological techniques. Alternatively, it is also envisaged that biomarkers in the respective tissue or organ of the patient are detected in order to control whether or not the therapeutic approach is effective. Additionally or alternatively, it is also possible to evaluate the general appearance of the respective patient, which will also aid the skilled practitioner to evaluate whether the therapy is effective. Those skilled in the art are aware of numerous other ways which will enable a practitioner to observe a therapeutic effect of the compound for use in the treatment of cell-based diseases as disclosed herein in the context of a method or use of the present invention.

While it is possible to administer the conjugates of the present invention directly without any formulation, in another aspect of the present invention the compounds are preferably employed in the form of a pharmaceutical or veterinary formulation composition, comprising a pharmaceutically or veterinary acceptable carrier or excipient and the conjugate of the present invention. Thus, according to another aspect the present invention also provides for a pharmaceutical composition comprising the conjugate described herein or produced by a method for the preparation of said conjugate as described elsewhere herein. Said pharmaceutical composition preferably comprises a pharmaceutical acceptable excipient, diluent or carrier. The term “pharmaceutical composition” relates to a composition for administration to a patient, preferably a human patient.

The carrier used in combination with the conjugates of the present invention is water-based and forms an aqueous solution. An oil-based carrier solution containing the compound of the present invention is an alternative to the aqueous carrier solution. Either aqueous or oil-based solutions further contain thickening agents to provide the composition with the viscosity of a liniment, cream, ointment, gel, or the like. Suitable thickening agents are well known to those skilled in the art. Alternative embodiments of the present invention can also use a solid carrier containing the conjugate of the present. This enables in alternative embodiments to be apply the conjugate via a stick applicator, patch, or suppository. The solid carrier further contains thickening agents to provide the composition with the consistency of wax or paraffin.

Pharmaceutical excipients according to the present invention include, by the way of illustration and not limitation, diluent, disintegrants, binding agents, adhesives, wetting agents, polymers, lubricants, gliands, substances added to mask or counteract a disagreeable texture, taste or odor, flavors, dyes, fragrances, and substances added to improve appearance of the composition. Acceptable excipients include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable excipients for soft gelatin capsules include vegetable oils, waxes, fats, semisolid and liquid polyols. Suitable excipients for the preparation of solutions and syrups include, without limitation, water, polyols, sucrose, invert sugar and glucose. Suitable excipients for injectable solutions include, without limitation, water, alcohols, polyols, glycerol, and vegetable oils. The pharmaceutical compositions can additionally include preservatives, solubilizers, stabilizers, wetting agents, emulsifiers, sweeteners, colorants, flavorings, buffers, coating agents, or antioxidants.

The compositions according to the present invention are preferably formulated in a unit dosage form, each dosage containing about 1 to about 500 mg, more usually about 5 to about 300 mg, of the active ingredient. The term “unit dosage form” as used herein refers to physically discreet units suitable as unitary dosages for human subjects or other mammals, each unit containing a predetermined quantity of active material calculated to produce the desired therapeutic effect, in association with a suitable pharmaceutical carrier. As used herein, a “dosage” refers to an amount of therapeutic agent administered to a patient. As used herein, a “daily dosage” refers to the total amount of therapeutic agent administered to a patient in a day.

The conjugates disclosed herein are also useful for treating cell-based diseases such as cancer and autoimmune diseases in domestic animals such as cats, dogs, rabbits, guinea pigs, cows, sheeps, and horses. Thus, the invention also provides a veterinary formulation comprising a conjugate for use in the treatment of a subject suffering from a cell-based disease as defined elsewhere herein together with a veterinary acceptable diluents or carrier. Such formulations include in particular ointments, pour-on formulations, spot-on formulations, dips, sprays, mousses, shampoos, collar, and powder formulations.

In alternative embodiments, in particular when treating cancer, the conjugates according to the present invention can be administered as an adjunct therapy to surgery and/or radiotherapy. “Adjunct therapy” means in this regard that the conjugates described herein will enhance or complete the therapeutic effect reached by surgery and/or radiotherapy. This means that combination of surgery and/or radiotherapy with the conjugates described herein leads to a more positive disease outcome as if surgery and/or radiotherapy are performed without an adjunct therapy comprising the conjugates of the present invention.

Moreover, the conjugates described herein can be administered in combination with another drug or compound used to treat a cell-based disease, such as cancer or an autoimmune disease. Said compound additionally combined with the conjugates described herein is no immunotoxin or conjugate comprising an immunotoxin. Many compounds are known to the skilled person to have positive effects on cell-base diseases and are therapeutically used in this regard. These drugs or compounds may be selected from the group consisting of an antihistamine, a glucocorticosteroid, a calcineurin inhibitor, a local anesthetic, serotonin-reuptake inhibitors (SSRI), menthol, camphor, neuroleptic, a topical antidepressant, a tetracyclic, a neurokinin-1 receptor antagonist, a mu-opioid receptor antagonists, a kappa opioid receptor antagonist, a protease inhibitor, a protease-activated receptor antagonist, a gastrin-realizing peptide, a gastrin-releasing peptide receptor antagonist, a brain-derived natriuretic peptide (BNP), a brain-derived natriuretic peptide (BNP) receptor antagonist, a dynorhin receptor antagonist, a cytokine, a cytokine antagonist, a quinolone, a chemokine receptor antagonist, and a botulinum toxin, just to name some. Said combination according to the present invention can be administered as a combined formulation or separate from each other. Moreover, the compounds for use in the treatment of a subject suffering from a cell-based disease, preferably cancer or an autoimmune disease, according to the present invention can be combined with ultraviolet radiation therapy. In some embodiments the ultraviolet radiation therapy is UVA radiation. In some embodiments the ultraviolet radiation therapy is UVB radiation. In some embodiments the ultraviolet radiation therapy is PUVA radiation.

As disclosed elsewhere herein, the Duotoxins of the present invention are characterized by comprising modified ADP-ribosylating toxin. Said toxins are characterized in (i) lack of a translocation domain, but maintenance of the furin cleavage site, and (ii) a catalytic active domain, wherein all lysine residues have been substituted by any other amino acid. The Duotoxins comprising Pseudomonas exotoxin A, are further characterized by (iii) an unusual C-terminal RDELK-motif “RDEL” (FIG. 2A). As described elsewhere herein for Pseudomonas exotoxin (PE) A, a respective toxin exhibiting the desired features can be recombinant produced and liked to a therapeutic agent. Accordingly, the present invention also provides for a method for the preparation of the conjugate according to the present invention, comprising cloning a nucleic acid encoding for a fusion protein comprising a binding domain and an adenosine diphosphate (ADP) ribosylating toxin into an expression vector, transforming a host cell with said expression vector, cultivating said transformed host cell in a nutrient medium, expressing the fusion protein, extracting the fusion protein from said host cell or medium, and conjugating the immunotoxin fusion protein by a linker to a therapeutic agent.

The nucleic acid used in this regard encodes for a fusion protein comprising a binding domain and an adenosine diphosphate (ADP) ribosylating toxin, wherein said ADP ribosylating toxin comprises from the amino terminus:
(i) a furin cleavage site, preferably comprising an amino acid sequence as shown in SEQ ID NO: 33 or an amino acid sequence having at least 85% identity to the amino acid sequence as shown in SEQ ID NO: 33 and the amino acid in position 6 is an arginine,
(ii) a catalytic domain, preferably comprising an amino acid sequence as shown in SEQ ID NO: 34 or an amino acid sequence having at least 85% identity to the amino acid sequence as shown in SEQ ID NO: 34, wherein lysine residues in the catalytic domain have been substituted by any other amino acid, and
(iii) the amino acid sequence as shown in SEQ ID NO: 36, preferably as shown in SEQ ID NO: 37, or
   (i) a catalytic domain, preferably comprising an amino acid sequence as shown in SEQ ID NO: 35 or an amino acid sequence having at least 85% identity to the amino acid sequence as shown in SEQ ID NO: 35, wherein lysine residues in the catalytic domain have been substituted by any other amino acid.

Preferably the ADP-ribosylating toxin comprises an amino acid sequence as shown in SEQ ID NO: 38. Said nucleic acid sequence encoding for the fusion protein described herein is preferably cloned into a vector. Accordingly, the present invention also provides for a vector comprising the nucleic acid as defined elsewhere herein. The term “vector” as used herein refers to a nucleic acid molecule into which the nucleic acid molecule of the invention encoding for a fusion protein comprising a binding domain and an adenosine diphosphate (ADP) ribosylating toxin may be inserted or cloned. The vector may have a linear, circular, or supercoiled configuration. Preferably said vector comprises a nucleic acid sequence which is a regulatory sequence operably linked to said nucleic acid sequence encoding for the fusion protein as defined herein. The term “regulatory sequence” refers to DNA sequences, which are necessary to effect the expression of coding sequences to which they are ligated. The nature of such control sequences differs depending upon the host organism. In prokaryotes, control sequences generally include promoter, ribosomal binding site, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

The term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In case the control sequence is a promoter, it is obvious for a skilled person that double-stranded nucleic acid is preferably used. Thus, the recited vector is preferably an expression vector.

An “expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in prokaryotes and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the PL, lac, trp or tac promoter in E. coli, and examples of regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements, which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the fusion protein to a cellular compartment or secreting it into the medium may be added to the coding sequence of the recited nucleic acid sequence and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Preferably, the expression control sequences will be eukaryotic promoter systems in vectors capable of transforming an eukaryotic host cells, but control sequences for prokaryotic hosts may also be used. Once the vector has been incorporated into the appropriate host, the host is maintained under conditions suitable for high level expression of the nucleotide sequences, and as desired, the collection and purification of the fusion protein encoded by the nucleic acid of the invention may follow.

Especially preferred is the use of a plasmid or a virus containing the coding sequence of the fusion protein comprising a binding domain and a adenosine diphosphate (ADP) ribosylating toxin according to the present invention, and genetically fused thereto an N-terminal FLAG-tag and/or C-terminal His-tag. Preferably, the length of said FLAG-tag is about 4 to 8 amino acids, most preferably 8 amino acids. An above described polynucleotide can be used to transform the host using any of the techniques commonly known to those of ordinary skill in the art. Furthermore, methods for preparing fused, operably linked genes and expressing them in, e.g., mammalian cells and bacteria are well-known in the art (Sambrook, loc cit.).

To generate a platform for the rapid cloning of Duotoxins for additional targets, a recognition sites for restriction enzymes on the expression plasmid may be incorporated, which are located between the variable antibody fragment (Fᵥ) and the constant fragment (F_{c1}), i.e. flanking the nucleic acid sequences encoding Fv and Fc (FIG. 2A), allowing a rapid “cut and paste” cloning strategy. Restriction enzymes are a class of endonucleases that occurs naturally in prokaryotic and eukaryotic organisms and is well known to those skilled in the art. These enzymes can be used in the laboratory to cleave DNA molecules in a specific and predictable manner. Thus, restriction enzymes have proved to be indispensable tools in modern genetic research. Over 3000 restriction enzymes have been studied in detail, and more than 600 of these are available commercially (Primrose 1994, ISBN 0-632-03712-1; Micklos et al. 1996, ISBN 0-8053-3040-2, and Massey & Kreuzer 2001, ISBN 1-55581-176-0). In view of the present invention, the used recognition sites are preferably a NheI, a NdeI, or a HindIII recognition site, but other recognition sites for restriction enzymes can be equally used in this regard and are known to those skilled in the art.

According to the disclosed method for the preparation of the conjugate according to the present invention, the described vector comprising the nucleic acid as defined elsewhere herein can be transformed into a host cell. Accordingly, the present invention also provides for a host cell vector transformed with a vector as defined elsewhere herein. The term “transformed” or “transformation” as use herein refers to any method suitable to deliver or transport nucleic acid to the host cell, such as transformation, transfection, transduction, electroporation, magnetofection, lipofection and the like, known to those skilled in the art. Said host cell may be produced by introducing the above described vector of the invention or the above described nucleic acid molecule of the invention into the host cell. The presence of at least one vector or at least one nucleic acid molecule in the host may mediate the expression of a gene encoding the above described immunotoxin comprising a binding domain fused to a ADP-ribosylating toxin. The described nucleic acid molecule or vector of the invention, which is introduced in the host cell may either integrate into the genome of the host cell or it may be maintained extrachromosomally.

The transformed hosts can be grown in fermentors and cultured according to techniques known in the art to achieve optimal cell growth. The recombinant produced immunotoxin of the present invention can then be isolated from the growth medium, cellular lysates, or cellular membrane fractions. The isolation and purification of the, e.g., microbially expressed fusion proteins of the-invention may be by any conventional means such as, for example, preparative chromatographic separations and immunological separations such as those involving the use of monoclonal or polyclonal antibodies directed, e.g., against a tag of the fusion protein. The conditions for the culturing of a host cell, which allow the expression are known in the art to depend on the host system and the expression system/vector used in such process. The parameters to be modified in order to achieve conditions allowing the expression of a recombinant peptide are known in the art. Thus, suitable conditions can be determined by the person skilled in the art in the absence of further inventive input. Once expressed, the immunotoxin of the invention can be purified according to standard procedures of the art, including ammonium sulfate precipitation, affinity columns, column chromatography, gel electrophoresis and the like (Scopes, “Protein Purification”, Springer-Verlag, N.Y. (1982)). Substantially pure fusion proteins of at least about 90 to 95% homogeneity are preferred, and 98 to 99% or more homogeneity are most preferred, for pharmaceutical uses. Once purified, partially or to homogeneity as desired, the immunotoxin of the invention may then be conjugated, i.e. chemically linked to a therapeutic agent as described elsewhere herein and therapeutically used.

The host cell can be any prokaryote or eukaryotic cell. The term “prokaryote” is meant to include all bacteria, which can be transformed with nucleic acid molecules for the expression of an immunotoxin of the invention. Prokaryotic hosts may include gram negative as well as gram positive bacteria such as, for example, E. coli, S. typhimurium, Serratia marcescens and Bacillus subtilis. E. coli is preferred in this regard. The term “eukaryotic” is meant to include yeast, higher plant, insect and preferably mammalian cells. Depending upon the host employed in a recombinant production procedure, the protein encoded by the nucleic acid of the present invention may be glycosylated or may be non-glycosylated. Preferably, said the host is a bacterium or an insect, fungal, plant or animal cell. It is particularly envisaged that the recited host may be a mammalian cell.

### Examples

The following examples illustrate the invention. These examples should not be construed as to limit the scope of this invention. The examples are included for purposes of illustration and the present invention is limited only by the claims. It will be clear to a skilled person in the art that the invention may be practiced in other ways than as particularly described in the present description and examples. Numerous modifications and variations of the present invention are possible in light of the above teachings and, therefore, are within the scope of the appended claims.

### Example 1: Synthesis Duotoxin Conjugates

The Pseudomonas exotoxin contains a receptor binding domain, a transport-related domain II, and the catalytically active domain III (FIG. 1). The receptor-binding domain is exchanged with the antigen-binding, variable fragment of an antibody resulting in the final immunotoxin. To chemically link a small molecule like the DM1 to a protein, distinct linker chemistries can be used, as described for example in Ross and Wolfe, Journal of Pharmaceutical Sciences 2016; 105(2): 391-397 or Creative® Biolabs, Cat. No: ADCsR-044CL, Product Information. Linker moieties have a reactive group which defines the target amino acid at the protein. Possible reactive targets are the amid side chain of lysines, the thiol side chain of Cysteines, or the carboxyl side chain of Glutamate or Aspartate. The linkers that attack the carboxyl group are not stable in the serum and are therefore not useful for the conjugation of DM1. The steps needed to link DM1 via Cysteines are chemically more complex and need several levels of controls. Therefore, the easiest and the most stable linking chemistry for DM1 is currently the amid-directed coupling via Lysines. In previous experiments, we linked fluorescent Alexa molecules (Invitrogen) to immunotoxins using Amid-directed reactions. Immunotoxins which carry Lysine-linked Alexa molecules are three to ten-fold less active (unpublished data). The amid-directed coupling possibly disrupts the activity of PE sterically. We therefore cloned a PE which lacks all lysines (Ks) (FIG. 2A). The Lysine-free PE was achieved by (i) deleting domain II, by introducing the two mutations K59QR and K606R, and by exchanging the REDLK with RDEL. These three steps result in immunotoxins with similar activity than the parental PE. Whether a molecule that has all three changes simultaneously remains active had not been tested before. To ensure enough Lysines for a successful coupling reaction, we added the constant region of the human IgG to the antibody moiety (FIG. 2A). Additionally, a novel restriction site between the Fv and the Fc was introduced to facilitate future exchange of antibodies by “cut and paste” cloning. The final molecule which consists of a CD22-targeting Fab and a PE without Ks, no domain II, and the C-terminal RDEL is termed “LMIT-2”. LMIT-2 and Moxe have comparable activity (FIG. 2B) and hence, can be used for amid-coupling of DM1. As an essential control we constructed LMIT-3 which is identical to LMIT-2 except for the Glutamate at position 553 which is mutated to Aspartate (E553D). The E553D mutation completely disrupts the ADP-ribosylation. As expected, LMIT-3 is inactive by itself but it can block the cytotoxic effects of LMIT-2 in a dose dependent manner (FIG. 2B). Because all plasmids are constructed so that each individual component can easily be exchanged by a “cut and paste” cloning strategy, we have produced a platform for the rapid generation of novel immunotoxins and therefore Duotoxins. For the conjugation reaction, reagents from Levana Bioscience are used as indicated below. A PD-10 column is equilibrated by applying 5 ml of Conjugation Buffer 1 (50 mM Potassium Phosphate/50 mM Sodium Chloride, pH 6.5, 2 mM EDTA) a total of 5 times. 2.5 ml immunotoxin solution containing desired amount of protein are loaded and eluted in fractions by adding buffer 1. The solution containing the immunotoxin is then concentrated to 11 mg/ml. A 10 mM SMCC-DM1 solution is prepared in DMA (Dimethylacedamide) and 20 μl thereof is added to a total of 2 μg immunotoxin in buffer 1 and the solution is briefly mixed by pipetting gently up and down. Incubate conjugation reaction at room temperature for 4 hours. Equilibrate another PD-10 column with 5 ml of Storage buffer as described above. The storage buffer is defined as the buffer that stabilizes the respective antibody fragment best and varies therefore. Load the conjugation reaction to the column by gravity flow and elute stepwise by adding storage buffer. The protein concentration is determined by standard methods e.g. a spectrophotometer.

### Example 2: Activity Test of Duotoxin Conjugates In Vitro

After DM1 coupling, the monovalent immunotoxins have full PE-activity and demonstrate a more consistent DM1-activity than bivalent IgG-MMAE Next, DM1 was coupled to LMLIT-2 via SMCC-linker following manufacturer's instructions. On average, 3.4 DM1 molecules were coupled per LMIT-2 molecule. LMIT-2-DM1 was tested against various cell lines (FIG. 3A). For most cell lines including KOPN-8, the activity of LMIT-2 and LMIT-2-DM1 was identical. This demonstrates that the coupling via SMCC-linker to the novel PE without Lysines does not disrupt activity of the PE. For some cells like Mino, the LMIT-2 by itself killed only up to 70% whereas LMIT-2-DM1 killed all the cells at high concentrations. Cells like DOHH-2 that are resistant to immunotoxins were sensitive to the LMIT-2-DM1. We have not found a cell line which is resistant to both, the PE and the DM1 in vitro. The data suggest that Duotoxins are either equally or more active than common immunotoxins against all cultured cells tested. Because the synergy of immunotoxins and paclitaxel is small or absent in vitro it was not expected to see an enhancement of LMIT-2 activity when DM1 is coupled. To achieve a molecule that is similar to LMIT-2 but demonstrates solely the DM1 activity, we coupled DM1 to LIMT-3 (the inactive E553D toxin variant). On average 3.6 DM1 molecules per LMIT-3 molecule were coupled. The activity of LMIT-3-DM1 was then compared with LMIT-2 and the published activity of Pinatuzumab vedotin (PV). In vitro, PV and LMIT-3-DM1 are on average 3 logs less active than LMIT-2. The activity of PV varies substantially over a 3-log concentration range. In contrast, the monovalent LMIT-3-DM1 has a more consistent activity than the PV which has two bindings sites for CD22, suggesting a more predictable cytotoxicity of Duotoxins than of PV. The molar activity against many cell lines of the IgG-based anti-CD22-MMAE Pinatuzumab vedotin, LMIT-2, and LMIT-3-DM1 are shown in FIG. 3B. The activity is tested by plating the above described cell lines at 20,000 cells per well in a 96-well plate. The immunotoxin or duotoxin is added at indicated concentrations in a three-fold serial dilution, the cells incubated in a humidified incubator for 72 hours. The cells are then collected by centrifugation, stained with 7-AAD and Annexin V and cell death determined by flow cytometry. The concentration at which 50% of the cells are killed (IC50) reflects the activity of a drug against a cell and is determined from the flow cytometry data by non-linear regression analysis.

### Example 3: Time-Dependency of LMIT-2-DM1-Activity In Vitro

Activity of LMIT-2-DM1 is exposure time dependent. Recent data show that immunotoxins are more active the longer a target cell is exposed. This is relevant because serum levels fall within three hours to inactive concentrations. By changing bolus dose administration to a continuous administration the activity of immunotoxin increases by more than ten-fold (Muller et al. 2017, Oncotarget; 8:30644-30655). Because LMIT-2, LMIT-2-DM1, and LMIT-3-DM1 are similar in size and composition, we can anticipate a half-life of approximately 20 minutes. As shown for immunotoxins, the activity of LMIT-3-DM1 depends on the time cells are exposed (FIG. 4). LMIT-3-DM1 needs more than 24 hours to kill the majority of Z-138, 201 PC, and JeKo-1 cells Because individually, the PE and the DM-1 component of Duotoxins need more than 24 hours to reach relevant cytotoxicity, but blood levels fall within three hours to inactive concentrations, the Duotoxins will be more active by continuous administration than by bolus doses. The activity of Duotoxins is determined as the activity of immunotoxins described in Example 2.

### Example 4: Synergy of Duotoxins in Xenograft Models

NSG mice were injected with 10 million JeKo-1 cells on day −14. On day 0, the mice were implanted with the respective pumps as: 60 μg/100 μl LMIT-2-DM1, LMIT-2, LMIT-3-DM1, nothing, or LMIT-2 whereas these letter mice received 25 mg/kg Paclitaxel on day 2, 2 days after pump implantation. Mice were followed daily and sacrificed on day 21 for bone marrow analysis. The bone marrow was extracted from each femor separately, the cells stained with anti-hu-CD20-FITC and analyzed by flow cytometry. Duotoxins show expected synergy in the JeKo-1 xenografts. Because bolus doses of immunotoxins combined with bolus dose of paclitaxel shows an approximately 2-fold increase in efficacy in vivo, we tested the efficacy of the bolus doses of Duotoxins in the previously established systemic JeKo-1 mouse model. JeKo-1 bearing mice were treated with three doses of LMIT-2, LMIT-3-DM1, or LMIT-2-DM1 (FIG. 5A). At treatment start on day 14, the JeKo-1 BM-infiltration was 20%. The JeKo-1 BM-infiltration of mice treated with three doses of LMIT-2 from day 14 increased two-fold, of mice treated with LMIT-3-DM1 increased more than three-fold. The Duotoxin however was significantly better than either agent alone in a dose-dependent manner. The data show that bolus doses of LMIT-2-DM1 are more active than bolus doses of LMIT-2 or LMIT-3-DM1. We conclude that DM1 enhances the immunotoxin as paclitaxel. For comparative purposes, mice were treated with immunotoxin conjugated either with DM1 or with MMAE as therapeutic agent (FIG. 5B). In this approach, equimolar immunotoxin was conjugated either with 200 nmol DM1 or with 200 nmol MMAE under identical conditions and 0.8 mg/kg of respective duotoxin or the immunotoxin only control was administered from day 14 QOD. Mice were sacrificed at day 21 and analyzed for JeKo-1 bone marrow infiltration. It could be shown that both, DM1 as well as MMAE can be used as conjugate for duotoxins because both achieve high synergy. Thus, in sum, Duotoxins can selectively target the strong synergistic effects of a microtubule modifier plus immunotoxin to a target cell while healthy cells are not affected from both, the PE and the paclitaxel-like DM1.

The present invention can also be characterized by the following items:
1. A conjugate comprising an immunotoxin linked to a therapeutic agent, wherein said immunotoxin comprises a binding domain fused to an adenosine diphosphate (ADP) ribosylating toxin.
2. The conjugate according to item 1, wherein said ADP-ribosylating toxin is Pseudomonas exotoxin (PE) A or diphtheria toxin.
3. The conjugate according to item 2, wherein said binding domain is an antibody or functional fragment thereof.
4. The conjugate according to item 3, wherein said functional fragment is selected from the group consisting of an antibody binding fragment Fab, a disulfide-stabilized Fv (dsFv), or a single-chain Fv (scFv).
5. The conjugate according to items 1 to 4, wherein said binding domain is capable of binding to a cell surface protein, preferably CD22, mesotheline (MSLN), fms like tyrosine kinase 3 (FLT-3), HER2/neu or CD138.
6. The conjugate according to items 1 to 5, wherein said binding domain comprises a VL region comprising CDR-L1, CDR-L2 and CDR-L3 and/or a VH region comprising CDR-H1, CDR-H2 and CDR-H3 selected from:
   (a) CDR-L1 as shown in SEQ ID NO: 1, CDR-L2 as shown in SEQ ID NO: 2 and CDR-L3 as shown in SEQ ID NO: 3 and/or CDR-H1 as shown in SEQ ID NO: 4, CDR-H2 as shown in SEQ ID NO: 5 and CDR-H3 as shown in SEQ ID NO: 6;
   (b) CDR-L1 as shown in SEQ ID NO: 7, CDR-L2 as shown in SEQ ID NO: 8 and CDR-L3 as shown in SEQ ID NO: 9 and/or CDR-H1 as shown in SEQ ID NO: 10, CDR-H2 as shown in SEQ ID NO: 11 and CDR-H3 as shown in SEQ ID NO: 12;
   (c) CDR-L1 as shown in SEQ ID NO: 13, CDR-L2 as shown in SEQ ID NO: 14 and CDR-L3 as shown in SEQ ID NO: 15 and/or CDR-H1 as shown in SEQ ID NO: 16, CDR-H2 as shown in SEQ ID NO: 17 and CDR-H3 as shown in SEQ ID NO: 18; and
   (d) CDR-L1 as shown in SEQ ID NO: 19, CDR-L2 as shown in SEQ ID NO: 20 and CDR-L3 as shown in SEQ ID NO: 21 and/or CDR-H1 as shown in SEQ ID NO: 22, CDR-H2 as shown in SEQ ID NO: 23 and CDR-H3 as shown in SEQ ID NO: 24.
7. The conjugate according to items 1 to 6, wherein said binding domain comprises a VL region and/or a VH region selected from:
   (a) a VL region as shown in SEQ ID NO: 25 and/or a VH region as shown in SEQ ID NO: 26;
   (b) a VL region as shown in SEQ ID NO: 27 and/or a VH region as shown in SEQ ID NO: 28;
   (c) a VL region as shown in SEQ ID NO: 29 and/or a VH region as shown in SEQ ID NO: 30; and
   (d) a VL region as shown in SEQ ID NO: 31 and/or a VH region as shown in SEQ ID NO: 32.
8. The conjugate according to items 1 to 7, wherein said therapeutic agent has cytostatic and/or cytotoxic activity.
9. The conjugate according to item 8, wherein said therapeutic agent is an anti-cancer drug.
10. The conjugate according to item 9, wherein said anti-cancer drug is a microtubule-targeting drug.
11. The conjugate according to item 10, wherein said drug is selected from mertansine and monomethyl auristatin (MMA).
12. The conjugate according to item 11, wherein said mertansine is myatansinoid (DM1).
13. The conjugate according to item 11, wherein said monomethyl auristatin is monomethyl auristatin E (MMAE) or monomethyl auristatin D (MMAD).
14. The conjugate according to items 1 to 13, wherein at least one molecule of the therapeutic agent, preferably 2, more preferably 3, 4, 5, 6, 7, or 8 molecules of the therapeutic agent are linked to one immunotoxin molecule.
15. The conjugate according to items 1 to 14, wherein the ADP-ribosylating toxin comprises from the amino terminus:
   (i) a furin cleavage site, preferably comprising an amino acid sequence as shown in SEQ ID NO: 33 or an amino acid sequence having at least 85% identity to the amino acid sequence as shown in SEQ ID NO: 33 and the amino acid in position 6 is an arginine,
   (ii) a catalytic domain, wherein the catalytic domain comprises an amino acid sequence as shown in SEQ ID NO: 34 or an amino acid sequence having at least 85% identity to the amino acid sequence as shown in SEQ ID NO: 34, wherein lysine residues in the catalytic domain have been substituted by any other amino acid, and
   (iii) the amino acid sequence as shown in SEQ ID NO: 36, preferably as shown in SEQ ID NO: 37, or
   (i) a catalytic domain, preferably comprising an amino acid sequence as shown in SEQ ID NO: 35 or an amino acid sequence having at least 85% identity to the amino acid sequence as shown in SEQ ID NO: 35.
16. The conjugate according to items 1 to 15, wherein the ADP-ribosylating toxin comprises an amino acid sequence as shown in SEQ ID NO: 38.
17. The conjugate according to items 1 to 16, wherein the therapeutic agent is linked to the immunotoxin by a linker.
18. The conjugate according to item 17, wherein said linker is a serum-stable linker.
19. The conjugate according to item 12 or items 14 to 18, wherein myatansinoid (DM1) is linked to the immunotoxin by succinimidyl 4-(N-maleimidomethyl)cyclohexane-1-carboxylate (SMCC).
20. The conjugate according to items 13 to 18, wherein monomethyl auristatin E (MMAE) and monomethyl auristatin D (MMAD)myatansinoid (DM1) are linked to the immunotoxin by a OSu-Glu-vc-PAB-Linker or a thiol-linker.
21. The conjugate according to any one of items 1 to 20 for use in a method of treating a subject suffering from a cell-based disease, preferably cancer or an autoimmune disease.
22. The conjugate for use according to item 221 wherein the cancer is selected from the group consisting of lymphoma, preferably B-cell malignancies, leukemia, ovarian cancer, breast cancer, lung cancer, prostate cancer, colon cancer, kidney cancer, pancreatic cancer, mesothelioma, lymphoma, liver cancer, urothelial cancer, stomach cancer, and cervical cancer.
23. The conjugate for use according to item 21, wherein the autoimmune disease is a B-cell mediated disease, preferably any one selected from the group consisting of psoriasis, rheumatoid arthritis, multiple sclerosis, Sjögren's syndrome, and Guillain-Barré syndrome.
24. The conjugate for use according to items 21 to 25, wherein said subject is a mammal.
25. The conjugate for use according to item 24, wherein said mammal is a mouse, a rat, a guinea a pig, a rabbit, a cat, a dog, a monkey, a horse or a human, preferably a human.
26. The conjugate for use according to items 21 to 25, wherein the use comprises administering a therapeutically effective amount of said conjugate to the subject.
27. The conjugate for use according to item 26, wherein said conjugate is continuously administered.
28. The conjugate for use according to item 26 or 27, wherein said conjugate is administered intratumorally, parenterally, subcutaneously, intravenously, intramuscularly, intraperitoneally, by intracavitary, intraarterially, transdermally, by application to mucous membranes, intrathecally, or intraarticulary.
29. The conjugate for use according to items 21 to 28, wherein said conjugate is administered as an adjunct therapy to surgery and/or radiotherapy.
30. The conjugate for use according to items 21 to 29, wherein said conjugate is administered in combination with a compound used to treat cancer or autoimmune diseases.
31. A nucleic acid encoding an immunotoxin comprising a binding domain fused to an adenosine diphosphate (ADP) ribosylating toxin, wherein said ADP ribosylating toxin comprises from the amino terminus:
   (i) a furin cleavage site, preferably comprising an amino acid sequence as shown in SEQ ID NO: 33 or an amino acid sequence having at least 85% identity to the amino acid sequence as shown in SEQ ID NO: 33 and the amino acid in position 6 is an arginine,
   (ii) a catalytic domain, preferably comprising an amino acid sequence as shown in SEQ ID NO: 34 or an amino acid sequence having at least 85% identity to the amino acid sequence as shown in SEQ ID NO: 34, wherein lysine residues in the catalytic domain have been substituted by any other amino acid, and
   (iii) the amino acid sequence as shown in SEQ ID NO: 36, preferably as shown in SEQ ID NO: 37, or
   (i) a catalytic domain, preferably comprising an amino acid sequence as shown in SEQ ID NOs: 35 or an amino acid sequence having at least 85% identity to the amino acid sequence as shown in SEQ ID NO: 35.
32. The nucleic acid according to item 31, wherein the ADP-ribosylating toxin comprises an amino acid sequence as shown in SEQ ID NO: 38.
33. The nucleic acid according to item 31 or 32, wherein the binding domain is an antibody or a functional fragment thereof.
34. The nucleic acid according to item 33, wherein said functional fragment is an antibody binding fragment Fab, a disulfide-stabilized Fv (dsFv), or a single-chain Fv (scFv).
35. The nucleic acid according to items 31 to 34, wherein said binding domain is capable of binding to a cell surface protein, preferably CD22, mesotheline (MSLN), fms like tyrosine kinase 3 (FLT-3), HER2/neu or CD138.
36. The nucleic acid according to items 31 to 35, wherein said binding domain comprises a VL region comprising CDR-L1, CDR-L2 and CDR-L3 and/or a VH region comprising CDR-H1, CDR-H2 and CDR-H3 selected from:
   (a) CDR-L1 as shown in SEQ ID NO: 1, CDR-L2 as shown in SEQ ID NO: 2 and CDR-L3 as shown in SEQ ID NO: 3 and/or CDR-H1 as shown in SEQ ID NO: 4, CDR-H2 as shown in SEQ ID NO: 5 and CDR-H3 as shown in SEQ ID NO: 6;
   (b) CDR-L1 as shown in SEQ ID NO: 7, CDR-L2 as shown in SEQ ID NO: 8 and CDR-L3 as shown in SEQ ID NO: 9 and/or CDR-H1 as shown in SEQ ID NO: 10, CDR-H2 as shown in SEQ ID NO: 11 and CDR-H3 as shown in SEQ ID NO: 12;
   (c) CDR-L1 as shown in SEQ ID NO: 13, CDR-L2 as shown in SEQ ID NO: 14 and CDR-L3 as shown in SEQ ID NO: 15 and/or CDR-H1 as shown in SEQ ID NO: 16, CDR-H2 as shown in SEQ ID NO: 17 and CDR-H3 as shown in SEQ ID NO: 18; and
   (d) CDR-L1 as shown in SEQ ID NO: 19, CDR-L2 as shown in SEQ ID NO: 20 and CDR-L3 as shown in SEQ ID NO: 21 and/or CDR-H1 as shown in SEQ ID NO: 22, CDR-H2 as shown in SEQ ID NO: 23 and CDR-H3 as shown in SEQ ID NO: 24.
37. The nucleic acid according to items 31 to 36, wherein said binding domain comprises a VL region and/or a VH region selected from:
   (a) a VL region as shown in SEQ ID NO: 25 and/or a VH region as shown in SEQ ID NO: 26;
   (b) a VL region as shown in SEQ ID NO: 27 and/or a VH region as shown in SEQ ID NO: 28;
   (c) a VL region as shown in SEQ ID NO: 29 and/or a VH region as shown in SEQ ID NO: 30; and
   (d) a VL region as shown in SEQ ID NO: 31 and/or a VH region as shown in SEQ ID NO: 32.
38. The nucleic acid according to items 34 to 37, further comprising restriction sites flanking the nucleic acid sequences encoding for Fv and Fc.
39. The nucleic acid according to item 38, wherein the restriction site between the nucleic acid sequences encoding for Fv and Fc is a NheI, a NdeI, or a HindIII restriction site.
40. A vector comprising the nucleic acid as defined in any one of items 31 to 39.
41. The vector according to item 40, wherein said vector further comprises a regulatory sequence, which is operably linked to the nucleic acid sequences as defined in any one of items 31 to 39.
42. The vector according to item 41, wherein said vector is an expression vector.
43. The vector according to items 40 to 42, wherein said vector is a plasmid.
44. A host cell transformed with a vector as defined in any one of items 40 to 43.
45. The host cell according to item 44, wherein said host cell is a prokaryotic or eukaryotic cell.
46. The host cell according to item 45, wherein said prokaryotic host cell is E. coli.
47. A method for the preparation of the conjugate according to items 1 to 20, comprising cloning a nucleic acid encoding for a fusion protein comprising a binding domain and a adenosine diphosphate (ADP) ribosylating toxin into an expression vector, transforming a host cell with said expression vector, cultivating said transformed host cell in a nutrient medium, expressing the fusion protein, extracting the fusion protein from said host cell or medium, and conjugating the immunotoxin fusion protein by a linker to a therapeutic agent.
48. The method according to item 47, wherein said nucleic acid is any one defined in items 31 to 39.
49. The method according to item 47 or 48, wherein said therapeutic agent has cytostatic and/or cytotoxic activity.
50. The method according to items 47 to 49, wherein said therapeutic agent is an anti-cancer drug.
51. The method according to item 50, wherein said anti-cancer drug is a microtubule-targeting drug.
52. The method according to item 51, wherein said drug is selected from mertansine and monomethyl auristatin (MMA).
53. The method according to item 52, wherein said mertansine is myatansinoid (DM1).
54. The method according to item 53, wherein said monomethyl auristatin is monomethyl auristatin E (MMAE) or monomethyl auristatin D (MMAD).
55. The method according to items 47 to 54, wherein said host cell is a prokaryotic or eukaryotic host cell.
56. The method according to item 55, wherein said prokaryotic host cell is E. coli.
57. A pharmaceutical composition comprising the conjugate according to any one of items 1 to 20 or produced according to the method of any one of items 47 to 56.
58. The pharmaceutical composition according to item 57, further comprising a pharmaceutically acceptable excipient or carrier.
59. A method for the treatment of a subject suffering from cancer or an autoimmune disease, the method comprising administering a therapeutically effective amount of the conjugate according to items 1 to 20 to a subject in need thereof.
60. The method according to item 59, wherein said therapeutically effective amount is sufficient to alleviate or heal said cancer or said autoimmune disease.
61. Use of a compound for the preparation of a medicament for treatment of a subject suffering from cancer or an autoimmune disease, wherein said compound is selected from the conjugate according to items 1 to 20.

1. A conjugate comprising an immunotoxin linked to a therapeutic agent, wherein said immunotoxin comprises a binding domain fused to an adenosine diphosphate (ADP) ribosylating toxin.

2. The conjugate according to claim 1, wherein said ADP-ribosylating toxin is Pseudomonas exotoxin (PE) A or diphtheria toxin.

3. The conjugate according to claim 2, wherein said binding domain is an antibody or functional fragment thereof.

4. The conjugate according to claim 3, wherein said functional fragment is selected from the group consisting of an antibody binding fragment Fab, a disulfide-stabilized Fv (dsFv), or a single-chain Fv (scFv).

5. The conjugate according to claim 1, wherein said binding domain is capable of binding to a cell surface protein selected from CD22, mesotheline (MSLN), fms like tyrosine kinase 3 (FLT-3), HER2/neu or CD138.

6. The conjugate according to claim 1, wherein said binding domain comprises a VL region comprising CDR-L1, CDR-L2 and CDR-L3 and/or a VH region comprising CDR-H1, CDR-H2 and CDR-H3 selected from:
(a) CDR-L1 as shown in SEQ ID NO: 1, CDR-L2 as shown in SEQ ID NO: 2 and CDR-L3 as shown in SEQ ID NO: 3 and/or CDR-H1 as shown in SEQ ID NO: 4, CDR-H2 as shown in SEQ ID NO: 5 and CDR-H3 as shown in SEQ ID NO: 6;
(b) CDR-L1 as shown in SEQ ID NO: 7, CDR-L2 as shown in SEQ ID NO: 8 and CDR-L3 as shown in SEQ ID NO: 9 and/or CDR-H1 as shown in SEQ ID NO: 10, CDR-H2 as shown in SEQ ID NO: 11 and CDR-H3 as shown in SEQ ID NO: 12;
(c) CDR-L1 as shown in SEQ ID NO: 13, CDR-L2 as shown in SEQ ID NO: 14 and CDR-L3 as shown in SEQ ID NO: 15 and/or CDR-H1 as shown in SEQ ID NO: 16, CDR-H2 as shown in SEQ ID NO: 17 and CDR-H3 as shown in SEQ ID NO: 18; and
(d) CDR-L1 as shown in SEQ ID NO: 19, CDR-L2 as shown in SEQ ID NO: 20 and CDR-L3 as shown in SEQ ID NO: 21 and/or CDR-H1 as shown in SEQ ID NO: 22, CDR-H2 as shown in SEQ ID NO: 23 and CDR-H3 as shown in SEQ ID NO: 24.

7. The conjugate according to claim 1, wherein said binding domain comprises a VL region and/or a VH region selected from:
(a) a VL region as shown in SEQ ID NO: 25 and/or a VH region as shown in SEQ ID NO: 26;
(b) a VL region as shown in SEQ ID NO: 27 and/or a VH region as shown in SEQ ID NO: 28;
(c) a VL region as shown in SEQ ID NO: 29 and/or a VH region as shown in SEQ ID NO: 30; and
(d) a VL region as shown in SEQ ID NO: 31 and/or a VH region as shown in SEQ ID NO: 32.

8. (canceled)

9. The conjugate according to claim 1, wherein said therapeutic agent is an anti-cancer drug.

10. The conjugate according to claim 9, wherein said anti-cancer drug is a microtubule-targeting drug.

11. The conjugate according to claim 10, wherein said anti-cancer drug is selected from mertansine and monomethyl auristatin (MMA).

12. The conjugate according to claim 11, wherein said mertansine is myatansinoid (DM1).

13. The conjugate according to claim 11, wherein said monomethyl auristatin is monomethyl auristatin E (MMAE) or monomethyl auristatin D (MMAD).

14. The conjugate according to claim 1, wherein at least 2 molecules of the therapeutic agent are linked to one immunotoxin molecule.

15. The conjugate according to claim 1, wherein the ADP-ribosylating toxin comprises from the amino terminus:
(i) a furin cleavage site, comprising an amino acid sequence as shown in SEQ ID NO: 33 or an amino acid sequence having at least 85% identity to the amino acid sequence as shown in SEQ ID NO: 33 and the amino acid in position 6 is an arginine,
(ii) a catalytic domain, wherein the catalytic domain comprises an amino acid sequence as shown in SEQ ID NO: 34 or an amino acid sequence having at least 85% identity to the amino acid sequence as shown in SEQ ID NO: 34, wherein lysine residues in the catalytic domain have been substituted by any other amino acid, and
(iii) the amino acid sequence as shown in SEQ ID NO: 36, or as shown in SEQ ID NO: 37, or
(i) a catalytic domain, comprising an amino acid sequence as shown in SEQ ID NO: 35 or an amino acid sequence having at least 85% identity to the amino acid sequence as shown in SEQ ID NO: 35.

16. The conjugate according to claim 1, wherein the ADP-ribosylating toxin comprises an amino acid sequence as shown in SEQ ID NO: 38.

17. (canceled)

18. (canceled)

19. The conjugate according to claim 12, wherein myatansinoid (DM1) is linked to the immunotoxin by succinimidyl 4-(N-maleimidomethyl)cyclohexane-1-carboxylate (SMCC).

20. The conjugate according to claim 13, wherein monomethyl auristatin E (MMAE) and monomethyl auristatin D (MMAD)myatansinoid (DM1) are linked to the immunotoxin by a OSu-Glu-vc-PAB-Linker or a thiol-linker.

21. (canceled)

22. The method according to claim 59, wherein the cancer is selected from the group consisting of lymphoma, leukemia, ovarian cancer, breast cancer, lung cancer, prostate cancer, colon cancer, kidney cancer, pancreatic cancer, mesothelioma, lymphoma, liver cancer, urothelial cancer, stomach cancer, and cervical cancer, and the autoimmune disease is a B-cell mediated disease selected from the group consisting of psoriasis, rheumatoid arthritis, multiple sclerosis, Sjögren's syndrome, and Guillain-Barré syndrome.

23-46. (canceled)

47. A method for the preparation of the conjugate according to claim 1, comprising cloning a nucleic acid encoding for a fusion protein comprising a binding domain and a adenosine diphosphate (ADP) ribosylating toxin into an expression vector, transforming a host cell with said expression vector, cultivating said transformed host cell in a nutrient medium, expressing the fusion protein, extracting the fusion protein from said host cell or medium, and conjugating the immunotoxin fusion protein by a linker to a therapeutic agent.

48-58. (canceled)

59. A method for the treatment of a subject suffering from cancer or an autoimmune disease, the method comprising administering a therapeutically effective amount of the conjugate according to claim 1 to a subject in need thereof.

60. (canceled)

61. (canceled)


SEQUENCE LISTING (49 sequences)

SEQ ID NO: 1 - protein, 6 aa, Mus musculus
QDISNY

SEQ ID NO: 2 - protein, 3 aa, Mus musculus
YTS

SEQ ID NO: 3 - protein, 9 aa, Mus musculus
QQGNTLPWT

SEQ ID NO: 4 - protein, 8 aa, Mus musculus
GFAFSIYD

SEQ ID NO: 5 - protein, 8 aa, Mus musculus
ISSGGGTT

SEQ ID NO: 6 - protein, 16 aa, Mus musculus
ARHSGYGTHWGVLFAY

SEQ ID NO: 7 - protein, 7 aa, Mus musculus
ASSSVSY

SEQ ID NO: 8 - protein, 3 aa, Mus musculus
DTS

SEQ ID NO: 9 - protein, 9 aa, Mus musculus
QQWSKHPLT

SEQ ID NO: 10 - protein, 8 aa, Mus musculus
GYSFTGYT

SEQ ID NO: 11 - protein, 8 aa, Mus musculus
ITPYNGAS

SEQ ID NO: 12 - protein, 12 aa, Mus musculus
ARGGYDGRGFDY

SEQ ID NO: 13 - protein, 12 aa, Mus musculus
QSLLNSGNQKNY

SEQ ID NO: 14 - protein, 3 aa, Mus musculus
GAS

SEQ ID NO: 15 - protein, 9 aa, Mus musculus
QNDHSYPLT

SEQ ID NO: 16 - protein, 8 aa, Mus musculus
GFSLTNYG

SEQ ID NO: 17 - protein, 7 aa, Mus musculus
IWSGGST

SEQ ID NO: 18 - protein, 16 aa, Mus musculus
ARKGGIYYANHYYAMD

SEQ ID NO: 19 - protein, 6 aa, Mus musculus
QDVNTA

SEQ ID NO: 20 - protein, 3 aa, Mus musculus
SAS

SEQ ID NO: 21 - protein, 9 aa, Mus musculus
QQHYTTPPT

SEQ ID NO: 22 - protein, 8 aa, Mus musculus
GFNIKDTY

SEQ ID NO: 23 - protein, 8 aa, Mus musculus
IYPTNGYT

SEQ ID NO: 24 - protein, 13 aa, Mus musculus
SRWGGDGFYAMDY

SEQ ID NO: 25 - protein, 107 aa, Mus musculus
MDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEI

SEQ ID NO: 26 - protein, 126 aa, Mus musculus
MEVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGTHWGVLFAYWGQGTLVTVSSAS

SEQ ID NO: 27 - protein, 111 aa, Mus musculus
MDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGCGTKLEIKRTVA

SEQ ID NO: 28 - protein, 125 aa, Mus musculus
MQVQLQQSGPELEKPGASVKISCKASGYSFTGYTMNWVKQSHGKCLEWIGLITPYNGASSYNQKFRGKATLTVDKSSSTAYMDLLSLTSEDSAVYFCARGGYDGRGFDYWGQGTTVTVSSASTKG

SEQ ID NO: 29 - protein, 118 aa, Mus musculus
MDIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKRTVA

SEQ ID NO: 30 - protein, 129 aa, Mus musculus
MQVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSSASTKG

SEQ ID NO: 31 - protein, 112 aa, Artificial Sequence
  humanized
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVA

SEQ ID NO: 32 - protein, 126 aa, Artificial Sequence
  humanized
MEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKG

SEQ ID NO: 33 - protein, 10 aa, Pseudomonas aeruginosa
RHRQPRGWEQ

SEQ ID NO: 34 - protein, 227 aa, Pseudomonas aeruginosa
GPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDASQPGKPP

SEQ ID NO: 35 - protein, 367 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACASCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPA

SEQ ID NO: 36 - protein, 4 aa, Artificial Sequence
  PE carboxy terminus
  MISC_FEATURE (2)..(3): X is Asp or Glu
RXXL

SEQ ID NO: 37 - protein, 4 aa, Artificial Sequence
  PE specific carboxy terminus
REDL

SEQ ID NO: 38 - protein, 232 aa, Pseudomonas aeruginosa
RHRQPRGWEQLGGSPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDREQAISALPDYASQPGRPPREDL

SEQ ID NO: 39 - protein, 638 aa, Pseudomonas aeruginosa
MHLTPHWIPLVASLGLLAGGSFASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 40 - protein, 203 aa, Artificial Sequence
  chimeric
MDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLS

SEQ ID NO: 41 - protein, 229 aa, Artificial Sequence
  chimeric
MEVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGTHWGVLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCKA

SEQ ID NO: 42 - protein, 464 aa, Artificial Sequence
  LMIT-2 HC full sequence
MEVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGTHWGVLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCKASGGRHRQPRGWEQLGGSPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDREQAISALPDYASQPGRPPREDL

SEQ ID NO: 43 - protein, 202 aa, Artificial Sequence
  chimeric
MDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLS

SEQ ID NO: 44 - protein, 225 aa, Artificial Sequence
  chimeric
MQVQLQQSGPELEKPGASVKISCKASGYSFTGYTMNWVKQSHGKCLEWIGLITPYNGASSYNQKFRGKATLTVDKSSSTAYMDLLSLTSEDSAVYFCARGGYDGRGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCKA

SEQ ID NO: 45 - protein, 209 aa, Artificial Sequence
  chimeric
MDIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLS

SEQ ID NO: 46 - protein, 229 aa, Artificial Sequence
  chimeric
MQVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCKA

SEQ ID NO: 47 - protein, 203 aa, Artificial Sequence
  humanized
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLS

SEQ ID NO: 48 - protein, 226 aa, Artificial Sequence
  humanized
MEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCKA

SEQ ID NO: 49 - protein, 3 aa, Artificial Sequence
  peptide linker
SGG